(19) United States
(12) Patent Application Publication
(10) Pub. No.: US 2016/0361309 A1
(43) Pub. Date: Dec. 15, 2016

(54) METHODS OF TREATING CANCER PATIENTS RESPONDING TO EZH2 INHIBITOR GSK126

(71) Applicant: GLAXOSMITHKLINE INTELLECTUAL PROPERTY (NO.2) LIMITED, Brentford Middlesex (GB)

(72) Inventors: Michael T. McCABE, Collegeville, PA (US); Carentha L. CREASY, Collegeville, PA (US)

(21) Appl. No.: 15/121,095

(22) PCT Filed: Feb. 26, 2015
(86) PCT No.: PCT/IB2015/051443

§ 371 Date: Aug. 24, 2016

Related U.S. Application Data
(60) Provisional application No. 61/945,063, filed on Feb. 26, 2014.

(51) Int. Cl. A61K 31/496; C12Q 1/68; G01N 33/574; A61K 45/06

(52) U.S. Cl. CPC A61K 31/496; A61K 45/06; C12Q 1/6886; G01N 33/57426; C12Q 2600/156; C12Q 2600/158; G01N 2333/91017

(57) ABSTRACT

Disclosed herein are methods of treating cancer in a human, where the methods include determining at least one of the following in one or more samples from the human: the presence or absence of an alanine to valine mutation at residue 687 (A687V) in EZH2 in a sample from the human; or the presence or absence of an increased level of H3K27me2 in a sample from the human as compared to a control; and administering to the human an effective amount of the EZH2 inhibitor GSK126 or a pharmaceutically acceptable salt thereof if the A687V mutation is present, or an increased level of H3K27me2 is not present, or both, in the one or more samples, which is indicative of an increased likelihood of increased response rate and/or prolonged progression free survival.

### FIELD

This invention relates to methods of treating cancer in a subject in need thereof.

### BACKGROUND

The expanding development and use of targeted therapies for cancer treatment reflects an increasing understanding of key oncogenic pathways, and how the targeted perturbation of these pathways corresponds to clinical response. Difficulties in predicting efficacy to targeted therapies is likely a consequence of the limited global knowledge of causal mechanisms for pathway deregulation (e.g. activating mutations, amplifications).

Pre-clinical translational research studies for oncology therapies focuses on determining what tumor type and genotypes are most likely to benefit from treatment. Treating selected patient populations may help maximize the potential of a therapy. Pre-clinical cellular response profiling of tumor models has become a cornerstone in development of novel cancer therapeutics. Efforts to predict clinical efficacy using cohorts of in vitro tumor models have been successful (e.g. EGFR inhibitors are selectively useful in those tumors harboring EGFR mutations). Thus, expansive panels of diverse tumor derived cell lines could recapitulate an ‘all comers’ efficacy trial; thereby identifying which histologies and specific tumor genotypes are most likely to benefit from treatment. Numerous specific molecular markers are now used to identify patients most likely to benefit in a clinical setting.

EZH2 (enhancer of zeste homolog 2; human EZH2 gene: Cardoso, C, et al; European J of Human Genetics, Vol. 8, No. 3 Pages 174-180, 2000) is the catalytic subunit of the Polycomb Repressor Complex 2 (PRC2) which functions to silence target genes by tri-methylating lysine 27 of histone H3 (H3K27me3). Histone H3 is one of the five main histone proteins involved in the structure of chromatin in eukaryotic cells. Featuring a main globular domain and a long N-terminal tail, Histones are involved with the structure of the nucleosomes, a ‘beads on a string’ structure. Histone proteins are highly post-translationally modified however Histone H3 is the most extensively modified of the five histones. The term “Histone H3” alone is purposely ambiguous in that it does not distinguish between sequence variants or modification state. Histone H3 is an important protein in the emerging field of epigenetics, where its sequence variants and variable modification states are thought to play a role in the dynamic and long term regulation of genes.

EZH2 inhibitors that are useful in treating cancer have been reported in PCT applications PCT/US2011/035336, PCT/US2011/035340, and PCT/US2011/035344, each of which are incorporated by reference herein; to the extent that these incorporated references conflict with the current application, this current application controls. It is desirable to identify genotypes that are more likely to respond to these compounds.

### SUMMARY OF THE INVENTION

Disclosed herein are methods of treating cancer in a human in need thereof, comprising determining at least one of the following in one or more samples from said human: the presence or absence of an alanine to valine mutation at residue 687 (A687V) in EZH2 in a sample from said human; or the presence or absence of an increased level of H3K27me2 in a sample from said human as compared to a control; and administering to said human an effective amount of an EZH2 inhibitor or a pharmaceutically acceptable salt thereof if the A687V mutation is present, or an increased level of H3K27me2 is not present, or both, in the one or more samples, wherein the EZH2 inhibitor is a compound of Formula (I):

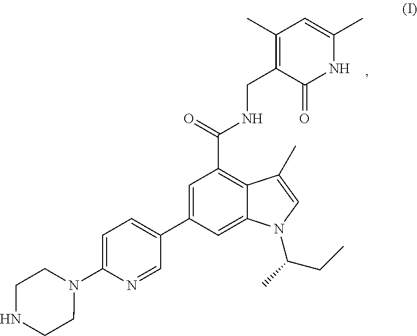 [SMILES: CC[C@H](C)n1cc(C)c2c(C(=O)NCc3c(C)cc(C)[nH]c3=O)cc(-c3ccc(N4CCNCC4)nc3)cc21]

or a pharmaceutically acceptable salt thereof.

Also disclosed are pharmaceutical compositions comprising an EZH2 inhibitor or a pharmaceutically acceptable salt thereof, for use in treating cancer in a human wherein at least a first sample from the human is determined to have an A687V mutation in EZH2, the absence of an increased level of level of H3K27me2 as compared to a control, or both, wherein the EZH2 inhibitor is a compound of Formula (I) as shown above, or a pharmaceutically acceptable salt thereof.

Also disclosed is an EZH2 nucleic acid or polypeptide sequence encoding an A687V mutation as a biomarker for use in cancer therapy with an EZH2 inhibitor having Formula (I) as shown above, o a pharmaceutically acceptable salt thereof.

Also disclosed are kits comprising kit for the treatment of cancer comprising a kit for determining one, two or three of: the presence or absence of an alanine to valine mutation at residue 687 (A687V) in EZH2 in a sample from said human; the presence or absence of an increased level of H3K27me2 as compared to a control; and the level of global H3K27me3 in a sample from the human as compared to a control; a means for determining one, two, or three of a, b, and c, and instructions for administering an EZH2 inhibitor, wherein said EZH2 inhibitor is an inhibitor of Formula I as shown above, or a pharmaceutically acceptable salt thereof, and wherein said instructions are in accordance with the methods disclosed herein.

### BRIEF DESCRIPTION OF THE DRAWINGS

FIG. 1. The A687V EZH2 mutation promotes increased activity with H3K27me1. A, EZH2 domain architecture (UniProt Q15910). The A687 residue and other known sites of activating mutations are highlighted within the SET domain. B, Alignment of human EZH2 with human EZH1, the fly ortholog E(z), and six other related SET domain-containing histone lysine methyltransferases showing that A687 is located immediately adjacent to the highly conserved NHS motif and is nearby the previously reported Y641 and A677 residues. Darker shading represents identical residues, and lighter shading represents conserved residues. In order for a column to be shaded, at least seven of nine residues must be conserved/identical. C, Comparison of biochemical activity across a library of 602 peptides representing sequences within histones H2a (blue diamonds), H2b (maroon squares), H3 (green triangles), or H4 (purple circles) for WT and A687V EZH2.

FIG. 2. Exogenous expression of A687V EZH2 stimulates trimethylation of H3K27 without diminishing H3K27me2. A, MCF-7 breast cancer cells were transiently transfected with mammalian expression constructs encoding either a WT or mutant form of EZH2. In addition, transfection reagent alone (No DNA) and empty vector treatments were included as controls. Cells were lysed 72 h after transfection, and whole-cell protein extracts were assessed for levels of H3K27me3, H3K27me2, H3K27me1, total histone H3, EZH2, and actin via western blotting. Actin and total histone H3 were included as loading controls. B, Average values of H3K27me3, H3K27me2, or H3K27me1 normalized to total histone H3 from at least two replicates. Values are presented as a percentage of the empty vector control sample.

FIG. 3. A B cell ALL cell line harboring A687V EZH2 exhibits elevated H3K27me3 levels. Western blot analysis was performed with antibodies specific for H3K27me3, H3K27me2, H3K27me1, total histone H3, EZH2, and actin using protein lysates from a panel of B cell ALL and DLBCL cell lines. Actin serves as a loading control. EZH2 mutation status as determined from full-length Sanger sequencing is indicated.

FIG. 4. A687V EZH2 mutant ALL is highly dependent on EZH2 activity. A, Sensitivity of ALL cell lines to EZH2 inhibition with GSK126. Cells were treated with a 20-point 2-fold dilution series of GSK126 (range 36 μM-70 pM) for 6 days and cell growth was measured using Cell Titer-Glo (Promega). Data are represented as the concentration of GSK126 required to inhibit 50% of growth (growth IC₅₀). B, Western blot analysis of H3K27me3, H3K27me2, H3K27me1, and total histone H3 following treatment of NALM-6 or SUP-B8 cell lines with varying concentrations of GSK126 for 72 hours.

FIG. 5. A687V EZH2 mutant cells exhibit growth inhibition and caspase activation in response to EZH2 inhibition. Temporal kinetics of GSK126-induced growth inhibition (A and C) and caspase 3/7 activation (B and D) in NALM-6 (A and B) and SUP-B8 (C and D) over a 6 day time course using Cell Titer-Glo or Caspase-Glo 3/7 (Promega), respectively.

FIG. 6. Activation of gene expression in A687V EZH2, but not WT EZH2, cells. A, Gene expression profiling of SUP-B8 and NALM-6 cells treated for 72 hours with 0.1% DMSO or 500 nM GSK126. Represented are 996 probe sets with significantly altered gene expression in either cell line following GSK126 treatment. Green, lower expression. Red, higher expression. B, Venn diagrams depicting the number of up- and down-regulated probe sets and their overlap across the cell lines. C, Summary of gene ontology terms enriched among the genes up-regulated with GSK126 in SUP-B8 or NALM-6 cells.

FIG. 7. EZH2 A687 coordinates an active site water molecule required for the stabilization of the H3K27me0 substrate. A homology model of EZH2 was generated using the crystal structure of GLP/EHMT1 bound to an H3K9me2 peptide substrate. Represented are (A) WT EZH2 with H3K27me0, (B) A687V EZH2 with H3K27me0, and (C) A687V EZH2 with H3K27me1.

FIG. 8. Levels of H3K27me2 and H3K27me3 in various B-cell ALL and DLBCL cell lines with and without mutations in EZH2.

### DETAILED DESCRIPTION OF THE INVENTION

EZH2 is the catalytically active methyltransferase component of the Polycomb Repressive Complex 2 (PRC2) and functions to methylate histone H3 on lysine 27 (H3K27) (Kuzmichev et al., 2002; Muller et al., 2002). Tri-methylation of H3K27 (H3K27me3) induces chromatin condensation and transcriptional repression of genes involved in development and differentiation (Bracken et al., 2006; Cao et al., 2002; Kirmizis et al., 2004). EZH2 is over-expressed in many human tumor types including prostate, breast, neuroendocrine lung, renal, and others (Findeis-Hosey et al., 2011; Kleer et al., 2003; Simon and Lange, 2008; Takawa et al., 2011; Varambally et al., 2002; Wagener et al., 2010). Diverse mechanisms underlie EZH2 over-expression including amplification (Bracken et al., 2003; Saramaki et al., 2006), E2F activation (Bracken et al., 2003), and loss of repressive microRNAs (miR-101, -26a) (Sander et al., 2008; Varambally et al., 2008). EZH2 is also recurrently mutated at specific residues in human non-Hodgkin lymphoma. EZH2 tyrosine 641 (Y641) is mutated in 14-22% of germinal center B cell (GCB) diffuse large B cell lymphomas (DLBCL) and 7-22% of follicular lymphomas (FL) (Bodor et al., 2011; Morin et al., 2010; Ryan et al., 2011). Additionally, alanine 677 of EZH2 is mutated to a glycine (A677G) in roughly 1-2% of DLBCLs (McCabe et al., 2012a; Morin et al., 2011).

These recurrent EZH2 mutations affect residues located within the lysine binding pocket that are critical for the positioning of the K27 substrate during the methyl transfer reaction. The lysine binding pocket of WT EZH2 is designed to stabilize the K27me0 substrate, but is also limited by steric hinderance with the larger K27me2 substrate. Mutations of Y641 to smaller residues (Y641N/F/S/H/C) relieve the steric hinderance with K27me2 thereby promoting the generation of H3K27me3, but this occurs at the expense of stabilizing the smaller H3K27me0 and me1 substrates (McCabe et al., 2012a; Sneeringer et al., 2010; Yap et al., 2011). Thus, while the Y641X EZH2 mutants have gained the ability to efficiently convert H3K27me2 into H3K27me3, they require coordination with the WT EZH2 as Y641X mutants possess little activity with an H3K27me0 substrate (Sneeringer et al., 2010).

The A677 residue of EZH2 is not located directly in the lysine binding pocket, but rather resides behind the Y641 residue (McCabe et al., 2012a). The mutation of A677 to the smaller glycine residue increases activity with an H3K27me2 substrate by enlarging the lysine binding pocket. Importantly, however, because this mutant retains the wild-type Y641 residue, it does not compromise the key interactions for positioning K27me0 and me1. Therefore, the A677G mutant is an efficient methyltransferase with any of the H3K27 substrates (H3K27me0/1/2) (McCabe et al., 2012a). These mutations provide a mechanism for lymphoma cells to dramatically increase global H3K27me3 and presumably permit aberrant transcriptional silencing of several genes involved in normal cellular homeostasis.

Recently, recurrent mutation of EZH2 A687 (footnote: residue numbering based on NM 001203247/NP 001190176; A692 in NM 004456/NP 004447) to a valine (A687V) was reported in large scale sequencing studies of non-Hodgkin lymphoma (Lohr et al., 2012; Morin et al., 2011). Biochemical characterization of A687V EZH2 demonstrated that this mutant exhibited a substrate preference unique from that of WT, Y641X, and A677G EZH2. A687V EZH2 exhibited greatly reduced activity with an H3K27me0 substrate, 4-fold increased activity with H3K27me1, and little change with H3K27me2 (Majer et al., 2012). Herein, we demonstrate that although A687V EZH2 does not have increased activity with H3K27me2 substrates, it promotes hyper-trimethylation of H3K27 and does so without dramatic depletion of H3K27me2 like other previously reported Y641 and A677 mutants. Additionally, we identify this mutation in a B-cell acute lymphoblastic leukemia (ALL) cell line possessing elevated H3K27me3 levels, and using GSK126 (McCabe et al., 2012b), a highly-specific inhibitor of EZH2 catalytic activity, we demonstrate that this A687V EZH2 mutant cell line is highly dependent on EZH2 function for survival.

One embodiment provides methods of treating cancer in a human in need thereof, comprising determining at least one of the following in a sample from said human:
a. the presence or absence of an alanine to valine mutation at residue 687 (A687V) in EZH2 in a sample from said human; or
b. the presence or absence of an increased level of H3K27me2 as compared to a control,
   and administering to said human an effective amount of an EZH2 inhibitor or a pharmaceutically acceptable salt thereof if the A687V mutation is present, or an increased level of H3K27me2 is not present, or both, in said sample. In yet a further embodiment of the method, the EZH2 inhibitor is a compound having Formula I or a pharmaceutically acceptable salt thereof as disclosed herein.

Another embodiment provides an EZH2 inhibitor or a pharmaceutically acceptable salt thereof, or a composition comprising the same, for use in treating cancer in a human wherein at least a first sample from the human is determined to have an A687V mutation in EZH2, the absence of an increased level of level of H3K27me2 as compared to a control, or both, wherein the EZH2 inhibitor is a compound of Formula I.

In any of the methods herein, the determinations of a and b, as well as any further determination or detection, e.g. of mutations or methylation status described herein, may be done in any order. In certain embodiments of the methods herein both a and b are determined or detected, in any order. In a further embodiment, one of a or b are determined or detected, and the presence or absence of an increased level of H3K27me3 as compared to a control is determined or detected, where any of the determinations can be made in any order.

In another embodiment, an EZH2 inhibitor, such as a compound described herein or a pharmaceutically acceptable salt thereof as described herein, is administered if it is determined that an A687V mutation and an increased level of H3K27me3 as compared to a control is present. In a further embodiment, the EZH2 inhibitor is a compound having Formula I or a pharmaceutically acceptable salt thereof as disclosed herein. In yet a further embodiment, the EZH2 inhibitor is a compound having Formula X. In yet a further embodiment, the EZH2 inhibitor is a compound having Formula I or a pharmaceutically acceptable salt thereof as disclosed herein.

Another embodiment provides an EZH2 inhibitor or a pharmaceutically acceptable salt thereof, or a composition comprising the same, for use in treating cancer in a human wherein at least a first sample from the human is determined to have an A687V mutation in EZH2 and the presence of an increased level of level of H3K27me3 as compared to a control, or both, wherein the EZH2 inhibitor is a compound of Formula X. Another embodiment provides an EZH2 inhibitor or a pharmaceutically acceptable salt thereof, or a composition comprising the same, for use in treating cancer in a human wherein at least a first sample from the human is determined to have an A687V mutation in EZH and the presence of an increased level of level of H3K27me3 as compared to a control, or both, wherein the EZH2 inhibitor is a compound of Formula I.

In another embodiment, an EZH2 inhibitor, such as a compound described herein or a pharmaceutically acceptable salt thereof as described herein, is administered if it is determined that there is not an increased level (e.g. an absence of an increased level) of H3K27me2 as compared to a control and there is an increased level of H3K27me3 as compared to a control. In yet a further embodiment, the EZH2 inhibitor is a compound having Formula X. In yet a further embodiment, the EZH2 inhibitor is a compound having Formula I or a pharmaceutically acceptable salt thereof as disclosed herein.

Another embodiment provides an EZH2 inhibitor or a pharmaceutically acceptable salt thereof, or a composition comprising the same, for use in treating cancer in a human wherein at least a first sample from the human is determined to have an absence of an increased level of H3K27me2 methylation and the presence of an increased level of level of H3K27me3 as compared to a control, wherein the EZH2 inhibitor is a compound of Formula X. In a further embodiment, the EZH2 inhibitor is a compound having Formula I or a pharmaceutically acceptable salt thereof as disclosed herein.

In another embodiment, an EZH2 inhibitor, such as a compound described herein or a pharmaceutically acceptable salt thereof as described herein, is administered if the following three determinations are made, in any order: 1) it is determined that an A687V mutation is present; 2) it is determined that there is not an increased level of H3K27me2; and 3) it is determined that there is an increased level of H3K27me3 as compared to a control. In yet a further embodiment, the EZH2 inhibitor is a compound having Formula X. In yet a further embodiment, the EZH2 inhibitor is a compound having Formula I or a pharmaceutically acceptable salt thereof as disclosed herein.

Another embodiment provides an EZH2 inhibitor or a pharmaceutically acceptable salt thereof, or a composition comprising the same, for use in treating cancer in a human wherein at least a first sample from the human is determined to have an A687V mutation in EZH2, and a sample from the human is determined to not have an increased level of H3K27me2; and a sample from the human is determined to have an increased level of H3K27me3 as compared to a control, wherein the EZH2 inhibitor is a compound of Formula X or a pharmaceutically acceptable salt thereof. In yet a further embodiment, the EZH2 inhibitor is a compound having Formula I or a pharmaceutically acceptable salt thereof as disclosed herein.

Another embodiment provides methods of treating cancer in a human in need thereof, comprising determining in a sample from said human the presence or absence of an alanine to valine mutation at residue 687 (A687V) in EZH2 in a sample from said human and administering to said human an effective amount of an EZH2 inhibitor or a pharmaceutically acceptable salt thereof if the A687V mutation is present in said sample, wherein the EZH2 inhibitor is a compound having Formula I or a pharmaceutically acceptable salt thereof as disclosed herein.

Another embodiment provides an EZH2 inhibitor or a pharmaceutically acceptable salt thereof, or a composition comprising the same, for use in treating cancer in a human wherein at least a first sample from the human is determined to have an A687V mutation in EZH2, wherein the EZH2 inhibitor is a compound of Formula I or a pharmaceutically acceptable salt thereof.

The present invention also provides methods of treating cancer in a human, where the method comprises the following steps: (a) determining the level of H3K27me2 from at least one sample (e.g. a tumor cell) from said human and (b) administering to said human an effective amount of an EZH2 inhibitor or a pharmaceutically acceptable salt thereof, e.g. in a pharmaceutical composition, if an increased level of H3K27me2 is not detected. In yet a further embodiment, the EZH2 inhibitor is a compound having Formula X. In yet a further embodiment, the EZH2 inhibitor is a compound having Formula I or a pharmaceutically acceptable salt thereof as disclosed herein.

In further embodiments, the level of H3K27me2 is detected and the H3K27me3 is detected, and an EZH2 inhibitor or a pharmaceutically acceptable salt thereof is administered if an increased level of H3K27me3 is detected and an increased level of H2K27me2 is not detected. In yet a further embodiment, the EZH2 inhibitor is a compound having Formula X. In yet a further embodiment, the EZH2 inhibitor is a compound having Formula I or a pharmaceutically acceptable salt thereof as disclosed herein.

In other embodiments, an increase in the level of global methylation of a cancer cell or tumor cell is determined. In other embodiments, the level of H3K27 methylation are determined. In other embodiments, the level of H3K27me0, H3K27me1, H3K27me2, and H3K27me3 are determined and an increase in the level of H3K27me3 suggests treatment with an EZH2 inhibitor. In further embodiments of the invention in this paragraph, the levels of methylation are compared to a control, and relative decrease in H3K27me2 methylation, relative to a control suggests treatment with an EZH2 inhibitor.

In further embodiments of any of the methods or uses herein, other EZH2 mutations known in the art are determined, either alone or in combination and in any order, with determining the presence or absence of an A687 mutation, e.g. A687V, or the presence or absence of an increased level of H3K27me2, or the presence or absence of an increased level of H3K27me3.

In one such embodiment of determining the presence of other EZH2 mutations, the presence or absence of a heterozygous Y641N mutation in a sample from a human in need of treatment for B-cell acute lymphoblastic leukemia, and if said mutation is present, then the human in need of treatment for B-cell acute lymphoblastic leukemia is treated with an EZH2 inhibitor or pharmaceutically acceptable salt thereof as described herein.

Methods of determining a mutation in EZH2, e.g. at A687, are well known to one of skill in the art and are described herein in the detailed description and Examples, and include identifying a patient based on a sample that is determined to have a mutation in EZH2 such as by PCR, such as qPCR, sequencing, such as next generation sequencing, microarray detection among other methods known in the art. Preferred methods of determining a mutation in EZH2 are: identifying a human based on a sample that was subject to PCR and detecting the mutation in a sample from the human. Methods of determining an increased or decreased level of methylation, e.g., H3K27me2 or H3K27Me3, relative to a control are well known in the art, including those shown in the Examples. A preferred assay for determining an increased level of methylation comprises performing western blot analysis with an antibody specific for me2 methylation of lysine 27 of Histone 3 or an antibody specific for me3 methylation of lysine 27 of Histone 3. A control can be any one of skill in the art would choose, such as a matched cell from a human, a matched tissue from a human, a cell of the same origin as the tumor but known to have wild type EZH2, or a devised control that correlates with what is seen in non-cancerous cells of the same origin or in cells with wild-type EZH2, such as a cell line. A preferred control is a cell of the same origin as that of the tumor that was sampled from the human.

In any embodiment of the invention, the sample comprises at least one cancer cell. In further embodiments certain such embodiments, the sample is a biological sample. One of skill in the art can identify a suitable biological sample for each cancer. For a blood cancer such as lymphoma, the biological sample is a blood sample. For solid tumors, the biological sample is a sample of the tumor such as a biopsy or a blood sample comprising cell-free DNA from the tumor.

In any one of the embodiments of the invention herein, the cancer is lymphoma. In further embodiments of the method of the invention, the lymphoma is selected from the group consisting of: B-cell acute lymphoblastic leukemia, germinal center B-cell (GCB), Diffuse Large B-cell Lymphoma (DLBCL), Splenic marginal zone lymphoma (SMZL), Waldenström's macroglobulinemia lymphoplasmacytic lymphoma (WM), Follicular lymphoma (FL), Mantle Cell Lymphoma (MCL), and Extra nodal marginal zone B-cell lymphoma of mucosa associated lymphoid tissue (MALT).

In embodiments of the methods of the invention herein, the A687V mutation is a somatic mutation.

In other embodiments of the methods of treating cancer as described herein, treatment, e.g. with an EZH2 inhibitor or a pharmaceutically acceptable salt thereof or any compound (including pharmaceutically acceptable salts) of the invention disclosed herein comprises an increased response rate and/or an improved progression free survival, as compared to an untreated human.

The present invention also relates to a method of treating cancer in a human which comprises the following steps: (a) performing a genotyping technique on a biological sample from the subject tumor to determine whether a somatic mutation of EZH2 at the alanine A687 residue is detected; and (b) correlating the detection of said mutations with increased likelihood of increased response rate and/or prolonged progression free survival when administered an EZH2 inhibitor. In further embodiments, the somatic mutation of EZH2 is an alanine to valine mutation at residue 687 (A687V).

The present invention also relates to a method of treating cancer in a human which comprises the following steps: (a) performing a genotyping technique on a biological sample from the subject tumor to determine whether said tumor has somatic mutations of EZH2 at the alanine residue at position 687 (A687); and (b) administering an effective amount of an EZH2 inhibitor or a pharmaceutically acceptable salt thereof to said human if said tumor has a mutation of EZH2 at the EZH2 at the alanine residue at position 687 (A687). In further embodiments, the somatic mutation of EZH2 is an alanine to valine mutation at residue 687 (A687V).

Also disclosed herein are kits useful for the treatment of cancer. One embodiment is a kit for determining one, two or three of: the presence or absence of an alanine to valine mutation at residue 687 (A687V) in EZH2 in a sample from said human; the presence or absence of an increased level of H3K27me2 as compared to a control; the level of global H3K27me3 in a sample from the human as compared to a control, a means for determining one, two, or three of a, b, and c, and instructions for administering an EZH2 inhibitor, wherein said EZH2 inhibitor is an inhibitor of Formula I or a pharmaceutically acceptable salt thereof, and wherein said instructions are in accordance with the methods and uses disclosed herein. In a further embodiment, the means is selected from the group consisting of primers, probes, and antibodies.

In any of the embodiments of the methods and uses described herein, the EZH2 inhibitor is in the form of a pharmaceutical composition.

### DEFINITIONS

The term “wild type” as is understood in the art refers to a polypeptide or polynucleotide sequence that occurs in a native population without genetic modification. As is also understood in the art, a “variant” includes a polypeptide or polynucleotide sequence having at least one modification to an amino acid or nucleic acid compared to the corresponding amino acid or nucleic acid found in a wild type polypeptide or polynucleotide, respectively. Included in the term variant is Single Nucleotide Polymorphism (SNP) where a single base pair distinction exists in the sequence of a nucleic acid strand compared to the most prevalently found (wild type) nucleic acid strand.

As used herein “genetic modification” or “genetically modified” or grammatical variations thereof refers to, but is not limited to, any suppression, substitution, amplification, deletion and/or insertion of one or more bases into DNA sequence(s). Also, as used herein “genetically modified” can refer to a gene encoding a polypeptide or a polypeptide having at least one deletion, substitution or suppression of a nucleic acid or amino acid, respectively. Genetic variants and/or SNPs can be identified by known methods. For example, wild type or SNPs can be identified by DNA amplification and sequencing techniques, DNA and RNA detection techniques, including, but not limited to Northern and Southern blot, respectively, and/or various biochip and array technologies. WT and mutant polypeptides can be detected by a variety of techniques including, but not limited to immunodiagnostic techniques such as ELISA and western Blot. As used herein, the process of detecting an allele or polymorphism includes but is not limited to serologic and genetic methods. The allele or polymorphism detected may be functionally involved in affecting an individual's phenotype, or it may be an allele or polymorphism that is in linkage disequilibrium with a functional polymorphism/allele. Polymorphisms/alleles are evidenced in the genomic DNA of a subject, but may also be detectable from RNA, cDNA or protein sequences transcribed or translated from this region, as will be apparent to one skilled in the art.

As is well known genetics, nucleotide and related amino acid sequences obtained from different sources for the same gene may vary both in the numbering scheme and in the precise sequence. Such differences may be due to numbering schemes, inherent sequence variability within the gene, and/or to sequencing errors. Accordingly, reference herein to a particular polymorphic site by number will be understood by those of skill in the art to include those polymorphic sites that correspond in sequence and location within the gene, even where different numbering/nomenclature schemes are used to describe them.

As used herein, “genotyping” a subject (or DNA or other sample) for a polymorphic allele of a gene(s) or a mutation in at least one polypeptide or gene encoding at least one polypeptide means detecting which mutated, allelic or polymorphic form(s) of the gene(s) or gene expression products (e.g., hnRNA, mRNA or protein) are present or absent in a subject (or a sample). Related RNA or protein expressed from such gene may also be used to detect mutant or polymorphic variation. As is well known in the art, an individual may be heterozygous or homozygous for a particular allele. More than two allelic forms may exist, thus there may be more than three possible genotypes. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g., sequencing, e.g., next generation sequencing (NGS)) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype or a tumor having at least one somatic mutation. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria.

As used herein, a human that is in need of treatment for cancer, may be “predisposed to” or “at increased risk of” a particular phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

A human that is in need of treatment for cancer may alternatively have a tumor or cancer cells with somatic mutations, and genotyping or other detection of the mutations can be performed.

As used herein “response” to treatment and grammatical variations thereof, includes but is not limited to an improved clinical condition of a patient after the patient received medication. Response can also mean that a patient's condition does not worsen upon that start of treatment. Response can be defined by the measurement of certain manifestations of a disease or disorder. With respect to cancer, response can mean, but is not limited to, a reduction of the size and or number of tumors and/or tumor cells in a patient. Response can also be defined by other endpoints such as a reduction or attenuation in the number of pre-tumorous cells in a patient.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

Samples, e.g. biological samples, for testing or determining of one or more mutations may be selected from the group of proteins, nucleotides, cellular blebs or components, serum, cells, blood, blood components, urine and saliva. Testing for mutations may be conducted by several techniques known in the art and/or described herein.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G12S” with reference to the K-ras polypeptide indicates that there is a glycine at amino acid number 12 of the wild-type K-ras sequence, and that glycine is replaced with a serine in the mutant K-ras sequence.

A “mutation” in a polypeptide or a gene encoding a polypeptide and grammatical variations thereof means a polypeptide or gene encoding a polypeptide having one or more allelic variants, splice variants, derivative variants, substitution variants, deletion variants, and/or insertion variants, fusion polypeptides, orthologs, and/or interspecies homologs. By way of example, at least one mutation of EZH2 would include an EZH2 in which part of all of the sequence of a polypeptide or gene encoding the polypeptide is absent or not expressed in the cell for at least one of the EZH2 proteins produced in the cell. For example, an EZH2 protein may be produced by a cell in a truncated form and the sequence of the truncated form may be wild type over the sequence of the truncate. A deletion may mean the absence of all or part of a gene or protein encoded by a gene. An EZH2 mutation also means a mutation at a single base in a polynucleotide, or a single amino acid substitution. Additionally, some of a protein expressed in or encoded by a cell may be mutated, e.g., at a single amino acid, while other copies of the same protein produced in the same cell may be wild type.

Mutations may be detected in the polynucleotide or translated protein by a number of methods well known in the art. These methods include, but are not limited to, sequencing, RT-PCR, and in situ hybridization, such as fluorescence-based in situ hybridization (FISH), antibody detection, protein degradation sequencing, etc. Epigenetic changes, such as methylation states, may also result in mutations and/or lack of expression of part or all of a protein from the corresponding polynucleotide encoding it.

As used herein “genetic abnormality” is meant a deletion, substitution, addition, translocation, amplification and the like relative to the normal native nucleic acid content of a cell of a subject. As used herein “gene encoding an EZH2 protein” means any part of a gene or polynucleotide encoding any EZH2 protein. Included within the meaning of this term are exons encoding EZH2. Gene encoding EZH2 proteins include but are not limited to genes encoding part or all of EZH2.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

As used herein “overexpressed” and “overexpression” and grammatical variations thereof means that a given cell produces an increased number of a certain protein relative to a normal cell. For instance, some tumor cells are known to overexpress Her2 or Erb2 on the cell surface compared with cells from normal breast tissue. Gene transfer experiments have shown that overexpression of HER2 will transform NIH 3T3 cells and also cause an increase in resistance to the toxic macrophage cytokine tumor necrosis factor. Hudziak et al., “Amplified Expression of the HER2/ERBB2 Oncogene Induces Resistance to Tumor Necrosis Factor Alpha in NIH 3T3 Cells”, Proc. Natl. Acad. Sci. USA 85, 5102-5106 (1988). Expression levels of a polypeptide in a particular cell can be effected by, but not limited to, mutations, deletions and/or substitutions of various regulatory elements and/or non-encoding sequence in the cell genome.

As used herein, “treatment” means any manner in which one or more symptoms associated with the disorder are beneficially altered. Accordingly, the term includes healing or amelioration of a symptom or side effect of the disorder or a decrease in the rate of advancement of the disorder.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Tumors may be hematopoietic tumor, for example, tumors of blood cells or the like. Specific examples of clinical conditions based on such a tumor include leukemia such as chronic myelocytic leukemia or acute myelocytic leukemia; myeloma such as multiple myeloma; lymphoma and the like.

The cancer may be any cancer in which an abnormal number of blast cells are present or that is diagnosed as a haematological cancer or dysplasia, such as leukemia, myeloid malignancy or myeloid dysplasia, including but not limited to, undifferentiated acute myelogenous leukemia, myeloblastic leukemia, myeloblastic leukemia, promyelocytic leukemia, myelomonocytic leukemia, monocytic leukemia, erythroleukemia and megakaryoblastic leukemia. In one aspect, the cancer is a myeloid malignancy cancer. In another aspect, the cancer is leukemia. The leukemia may be acute lymphocytic leukemia, acute non-lymphocytic leukemia, acute myeloid leukemia (AML), chronic lymphocytic leukemia, chronic myelogenous (or myeloid) leukemia (CIVIL), and chronic myelomonocytic leukemia (CMML). In one embodiment, the human has agnogenic myeloid metaplasia and/or poor-risk myelodysplasia (MDS). In some aspects the cancer is relapsed or refractory.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In some embodiments, the sample is selected from the group consisting of cancer cells, tumor cells, cells, blood, blood components, urine and saliva.

### Compounds

In certain embodiments of the methods of treating cancer in a human in need thereof, the EZH2 inhibitor is of Formula X:

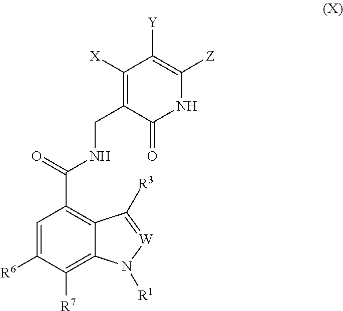

wherein:

W is N or CR²;

X and Z are each independently selected from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halogen, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};

Y is hydrogen or halogen;

R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, or —CONR^{a}NR^{a}R^{b};

When present R² is hydrogen, (C₁-C₈)alkyl, trifluoromethyl, alkoxy, or halogen, in which said (C₁-C₈)alkyl may be substituted with one to two groups selected from amino and (C₁-C₃)alkylamino;

R⁷ is hydrogen, (C₁-C₃)alkyl, or alkoxy; R³ is hydrogen, (C₁-C₈)alkyl, cyano, trifluoromethyl, —NR^{a}R^{b}, or halogen;

R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};

wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halogen, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halogen, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};
each R^{c} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};

R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or SO₂N((C₁-C₄)alkyl)((C₁-C₄)alkyl);

or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2, or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;

or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;

or a pharmaceutically acceptable salt thereof.

Compounds of Formula X, and methods of making the same are disclosed in WO2011/140324, which is incorporated by reference in its entirety herein.

In a further embodiment, EZH2 inhibitor is a compound of Formula (X) wherein W is CR²′ or a pharmaceutically acceptable salt thereof.

In yet a further embodiment, the EZH2 inhibitor is a compound having Formula I:

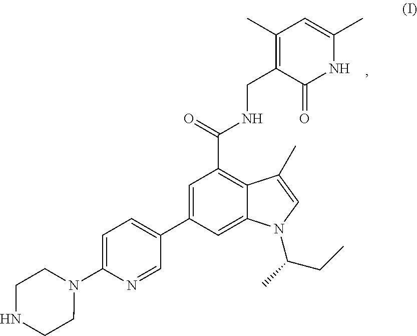 [SMILES: CC[C@H](C)n1cc(C)c2c(C(=O)NCc3c(C)cc(C)[nH]c3=O)cc(-c3ccc(N4CCNCC4)nc3)cc21]

or a pharmaceutically acceptable salt thereof. An EZH2 compound having Formula I or a pharmaceutically acceptable salt thereof is also referred to as GSK126, e.g. in the Examples herein.

Compounds having Formula I and methods of making the same are disclosed in WO 2011/140324, e.g. Example 270.

In another embodiment, the EZH2 inhibitor is Compound A having formula 1-(1-methylethyl)-N-[(6-methyl-2-oxo-4-propyl-1,2-dihydro-3-pyridinyl)methyl]-6-[6-(4-methyl-1-piperazinyl)-3-pyridinyl]-1H-indazole-4-carboxamide.

In another embodiment, the EZH2 inhibitor is Compound C having formula 1-Isopropyl-N-[(6-methyl-2-oxo-4-propyl-1,2-dihydro-3-pyridinyl)methyl]-6-[2-(4-methyl-1-piperazinyl)-4-pyridinyl]-1H-indazole-4-carboxamide

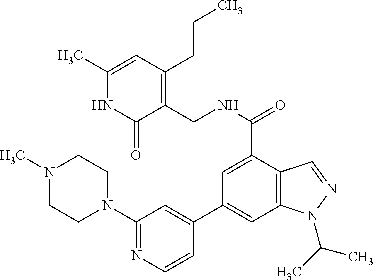 [SMILES: CCCc1cc(C)[nH]c(=O)c1CNC(=O)c1cc(-c2ccnc(N3CCN(C)CC3)c2)cc2c1cnn2C(C)C]

Additional EZH2 inhibitors are well known in the art. For example, EZH2 inhibitors are disclosed in WO 2011/140324, WO 2011/140325 and WO 2012/075080, each of which is incorporated by reference herein in its entirety. In any of the embodiments herein, the EZH2 inhibitor may be a compound disclosed in WO 2011/140324, WO 2011/140325 or WO 2012/075080.

One of skill in the art knows how to formulate EZH2 inhibitors, including those of Formula X and Formula I, into pharmaceutical preparations that include excipients and carriers, for example in an I.V. formulation, that are known in the art.

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁C₈alkyl” refers to an alkyl group having at least 1 and up to 8 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, and n-octyl and branched analogs of the latter 5 normal alkanes.

The term “alkoxy” as used herein means —O(C₁C₈alkyl) including —OCH₃, —OCH₂CH₃ and —OC(CH₃)₃ and the like per the definition of alkyl above.

The term “alkylthio” as used herein is meant —S(C₁C₈alkyl) including —SCH₃, —SCH₂CH₃ and the like per the definition of alkyl above.

The term “acyloxy” means —OC(O)C₁C₈alkyl and the like per the definition of alkyl above.

“Acylamino” means-N(H)C(O)C₁C₈alkyl and the like per the definition of alkyl above.

“Aryloxy” means —O(aryl), —O(substituted aryl), —O(heteroaryl) or —O(substituted heteroaryl).

“Arylamino” means —NH(aryl), —NH(substituted aryl), —NH(heteroaryl) or —NH(substituted heteroaryl), and the like.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

“Haloalkyl” refers to an alkyl group that is substituted with one or more halogen substituents, suitably from 1 to 6 substituents. Haloalkyl includes trifluoromethyl.

When “cycloalkyl” is used it refers to a non-aromatic, saturated, cyclic hydrocarbon ring containing the specified number of carbon atoms. So, for example, the term “C₃-C₈cycloalkyl” refers to a non-aromatic cyclic hydrocarbon ring having from three to eight carbon atoms. Exemplary “C₃-C₈cycloalkyl” groups useful in the present invention include, but are not limited to, cyclopropyl, cyclobutyl, cyclopentyl, cyclohexyl, cycloheptyl and cyclooctyl.

The term “C₅C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃C₈heterocycloalkyl” is used, it means a non-aromatic heterocyclic ring containing the specified number of ring atoms being, saturated or having one or more degrees of unsaturation and containing one or more heteroatom substitutions independently selected from O, S and N. Such a ring may be optionally fused to one or more other “heterocyclic” ring(s) or cycloalkyl ring(s). Examples are given herein below.

“Aryl” refers to optionally substituted monocyclic or polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl, and the like, as further illustrated below.

“Heteroaryl” means an optionally substituted aromatic monocyclic ring or polycarbocyclic fused ring system wherein at least one ring complies with Hückel's Rule, has the specified number of ring atoms, and that ring contains at least one heteratom independently selected from N, O and S. Examples of “heteroaryl” groups are given herein below.

The term “optionally” means that the subsequently described event(s) may or may not occur, and includes both event(s), which occur, and events that do not occur.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

### Pharmaceutical Formulations

While it is possible that, the compound of the present invention, as well as pharmaceutically acceptable salts and solvates thereof, may be administered as the raw chemical, it is also possible to present the active ingredient as a pharmaceutical composition. Accordingly, embodiments of the invention further provide pharmaceutical compositions, which include therapeutically effective amounts of a compound of Formula (I), or Compound A, or Compound B and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of Formula I, Compound A, or Compound B with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 800 mg, of a compound of the formula (I) depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known by one of skill in the art, e.g. in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

Dosage unit forms can also be in the form for i.v. delivery, of which one of skill in the art is capable of providing.

Dosage unit forms, e.g. for i.v. delivery, can also be in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines or other forms familiar to one of skill in the art.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of formula (I) or a pharmaceutically acceptable salt thereof will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) or a salt thereof for the treatment of a cancerous condition such as those described herein will generally be in the range of 0.1 to 100 mg/kg body weight of recipient (mammal) per day and more usually in the range of 1 to 12 mg/kg body weight per day. Thus, for a 70 kg adult mammal, the actual amount per day would usually be from 70 to 840 mg and this amount may be given in a single dose per day or more usually in a number (such as two, three, four, five or six) of sub-doses per day such that the total daily dose is the same. An effective amount of a salt or solvate thereof may be determined as a proportion of the effective amount of the compound of formula (I) per se. It is envisaged that similar dosages would be appropriate for treatment of the other conditions referred to above.

The amount of administered or prescribed compound according to these aspects of the present invention will depend upon a number of factors including, for example, the age and weight of the patient, the precise condition requiring treatment, the severity of the condition, the nature of the formulation, and the route of administration. Ultimately, the amount will be at the discretion of the attendant physician.

### Combinations and Additional Anti-Neoplastic Agents

In certain embodiments, the methods of the present invention further comprise administering one or more additional anti-neoplastic agents.

When an EZH2 inhibitor such as, but not limited to, Formula I, Compound A, or Compound B, is administered for the treatment of cancer, the term “co-administering” and derivatives thereof as used herein is meant either simultaneous administration or any manner of separate sequential administration of an EZH2 inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. If the administration is not simultaneous, the compounds are administered in a close time proximity to each other. Furthermore, it does not matter if the compounds are administered in the same dosage form, e.g. one compound may be administered topically or intravenously (i.v.) and another compound may be administered orally.

Typically, any anti-neoplastic agent that has activity versus a susceptible tumor or cancer (e.g. lymphoma) being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, any treatment for lymphoma, such as: R-CHOP, the five component treatment for non-Hodgkin's lymphoma, comprising: Rituximab, Cyclophosphamide, a DNA alkylating cross-linking agent; Hydroxydaunorubicin (i.e. doxorubicin or Adriamycin), a DNA intercalating agent; Oncovin (vincristine), which inhibits cell division by binding to the protein tubulin, and the corticosteroids Prednisone or prednisolone; CHOP, R-CVP (similar to R-CHOP, comprises rituximab, cyclophosphamide, vincristine, and prednisolone/prednisone), CVP; bortezomib; bendamustin; alemtuzumab; and radioimmunotherapy (e. ibritumomab (Zevalin), tositumomab (Bexxar)).

Other typical anti-neoplastic agents useful in the present invention include, but are not limited to. Class I and Class II histone deacetylase (HDAC) inhibitors (e.g., vorinostat), DNA methylase inhibitors (e.g. decitabine or azacitidine), histone acetyltransferase (HAT) inhibitors (e.g. p300 and PCAF inhibitors), anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present EZH2 inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL™. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)— N-carboxy-3-phenylisoserine, N-tert-butyl ester, 13-ester with 5β-20-epoxy-1,2α, 4,7β, 10β, 13α-hexahydroxytax-11-en-9-one 4-acetate 2-benzoate, trihydrate; is commercially available as an injectable solution as TAXOTERE™. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN™ as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN™ as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE™), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL™ as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN™ as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN™. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN™. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN™ tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN™ TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU™. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome™. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN™. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME™ or as an injectable as CERUBIDINE™. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX™ or ADRIAMYCIN RDF™. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE™. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID™ and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON™ and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U™ and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL™. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID™. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′, 2′-difluorocytidine monohydrochloride (β-isomer), is commercially available as GEMZAR™. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR™.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN′. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula F following, currently under development, including the racemic mixture (R,S) form as well as the R and S enantiomers:

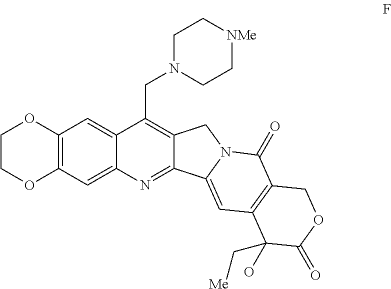 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Letrozole (trade name Femara) is an oral non-steroidal aromatase inhibitor for the treatment of hormonally-responsive breast cancer after surgery. Estrogens are produced by the conversion of androgens through the activity of the aromatase enzyme. Estrogens then bind to an estrogen receptor, which causes cells to divide. Letrozole prevents the aromatase from producing estrogens by competitive, reversible binding to the heme of its cytochrome P450 unit. The action is specific, and letrozole does not reduce production of mineralo- or corticosteroids.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and Bennett, C. F. and Cowsert, L. M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin™ erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Pazopanib which commercially available as VOTRIENT™ is a tyrosine kinase inhibitor (TKI). Pazopanib is presented as the hydrochloride salt, with the chemical name 5-[[4-[(2,3-dimethyl-2H-indazol-6-yl)methylamino]-2-pyrimidinyl]amino]-2-methylbenzenesulfonamide monohydrochloride. Pazoponib is approved for treatment of patients with advanced renal cell carcinoma.

Bevacisumab which is commercially available as AVASTIN™ is a humanized monoclonal antibody that blocks VEGF-A. AVASTIN™ is approved form the treatment of various cancers including colorectal, lung, breast, kidney, and glioblastomas.

mTOR inhibitors include but are not limited to rapamycin (FK506) and rapalogs, RAD001 or everolimus (Afinitor), CCI-779 or temsirolimus, AP23573, AZD8055, WYE-354, WYE-600, WYE-687 and Pp121.

Everolimus is sold as Afinitor® by Novartis and is the 40-O-(2-hydroxyethyl) derivative of sirolimus and works similarly to sirolimus as an mTOR (mammalian target of rapamycin) inhibitor. It is currently used as an immunosuppressant to prevent rejection of organ transplants and treatment of renal cell cancer. Much research has also been conducted on everolimus and other mTOR inhibitors for use in a number of cancers. It has the following chemical structure (formula V) and chemical name:

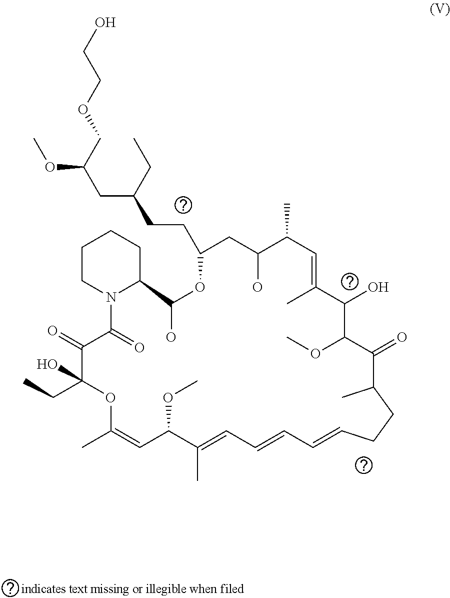

dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl) ethenyl] benzoic acid. Bexarotene is used to treat cutaneous T-cell lymphoma (CTCL, a type of skin cancer) in people whose disease could not be treated successfully with at least one other medication.

Sorafenib marketed as Nexavar™ is in a class of medications called multikinase inhibitors. Its chemical name is 4-[4-[[4-chloro-3-(trifluoromethyl)phenyl]carbamoylamino] phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Agents used in immunotherapeutic regimens may also be useful in combination with the compounds of formula (X) and formula I. There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by Formula VI, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

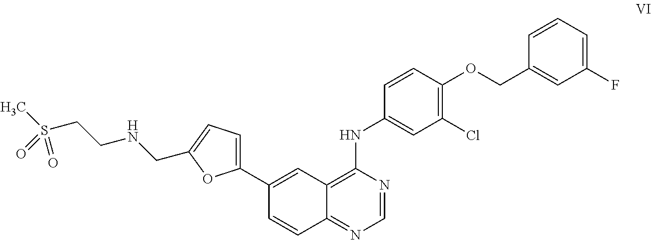 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (VI) may be prepared according to the procedures disclosed in WO 99/35146, published Jul. 15, 1999; and WO 02/02552 published Jan. 10, 2002.

Erlotinib, N-(3-ethynylphenyl)-6,7-bis{[2-(methyloxy)ethyl]oxy}-4-quinazolinamine Commercially available under the tradename Tarceva) is represented by formula VII, as illustrated:

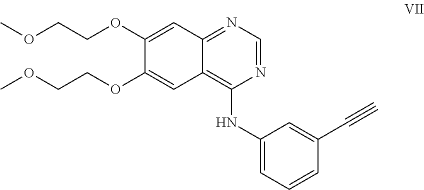 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine,N-(3-chloro-4-fluorophenyl)-7-methoxy-6-[3-4-morpholin)propoxy] is represented by formula VIII, as illustrated:

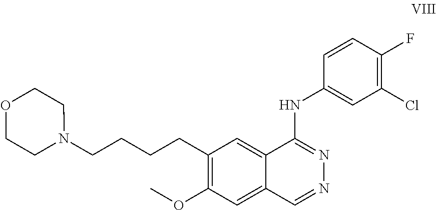 [SMILES: COc1cc2cnnc(Nc3ccc(F)c(Cl)c3)c2cc1CCCCN1CCOCC1]

Gefitinib, which is commercially available under the trade name IRESSA™ (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

Trastuzumab (HEREPTIN™) is a humanized monoclonal antibody that binds to the HER2 receptor. It original indication is HER2 positive breast cancer.

Cetuximab (ERBITUX™) is a chimeric mouse human antibody that inhibits epidermal growth factor receptor (EGFR).

Pertuzumab (also called 2C4, trade name Omnitarg) is a monoclonal antibody. The first of its class in a line of agents called “HER dimerization inhibitors”. By binding to HER2, it inhibits the dimerization of HER2 with other HER receptors, which is hypothesized to result in slowed tumor growth. Pertuzumab is described in WO01/00245 published Jan. 4, 2001.

Rituximab is a chimeric monoclonal antibody which is sold as RITUXAN™ and MABTHERA™. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA™. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

Any of the cancer treatment methods of the claimed invention may further comprise treatment with at least one additional anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors if one of a mutation in EZH2 at Y641 or A677 or an increased level of H3K27me3 is detected.

### EXAMPLES

### Example 1

### Structural Modeling of EZH2

A homology model of EZH2 was built using GLP/EHMT1 bound to an H3K9me2 peptide substrate (Protein Data Bank ID=2RFI) as a primary template and structurally compared to other related SET domain containing histone lysine methyltransferases with determined crystal structures as previously described (McCabe et al., 2012a).

### Example 2

### Cloning, Expression, and Purification of 5-Member PRC2 Complexes

Preparation of 5-member PRC2 complexes has previously been described (McCabe et al., 2012a). For A687V EZH2, human EZH2 in pENTR/TEV/D-TOPO was mutagenized by site-directed mutagenesis (QuikChange II XL, Agilent Technologies), the entire coding region of all mutants was confirmed by double-stranded DNA sequencing, and sub-cloned into pDEST8 with an N-terminal FLAG epitope tag. Individual baculovirus stocks were generated for expression of EED, SUZ12, RbAp48, AEBP2, and FLAG-tev-EZH2 and PRC2 complexes were purified using anti-FLAG M2 resin (Sigma) as previously described (McCabe et al., 2012a). For mammalian expression studies, WT human EZH2 was sub-cloned into pIRES2-ZsGreen1 (Clontech) and site-directed mutagenesis was utilized as described above to obtain the A687V mutant. All components and EZH2 mutations were confirmed by peptide mapping analysis.

### Example 3

### Biochemical Evaluation of Methyltransferase Activity

Unless otherwise stated, all reagents were obtained from Sigma and were at a minimum of reagent grade. Peptides contained within the peptide library were acquired from 21^{st} Century Biochemicals, AnaSpec (Fremont, Calif.), or Alta Bioscience (Birmingham, UK). Library peptides all contain a terminal biotin tag and range in purity from crude to 97%. Streptavidin SPA bead (RPNQ0261) and [³H]—S-adenosyl-methionine (SAM) were purchased from PerkinElmer.

All reactions were evaluated at ambient temperature in assay buffer containing 50 mM Tris-HCl (pH 8.0), 2 mM MgCl₂, 4 mM DTT, and 0.001% Tween-20. For a peptide library screen was run as 10 μL reactions in Greiner 384-well plates that were pre-stamped with 100 nL peptide (1 μM final) in 100% dimethyl sulfoxide (DMSO). [³H]-SAM (200 nM, 0.016 μCi/mL final) was added to the plate followed by the addition of WT or mutant PRC2 (16 nM final). Reactions were quenched after 1 hour via the addition of unlabeled SAM and streptavidin-coated SPA imaging beads at 0.5 mM and 1.5 mg/mL final concentrations, respectively. Activity was analyzed by reading the plates on a Wallac ViewLux CCD Imager (613/55 emission filter).

### Example 4

### Cell Culture

The SUP-B8 acute lymphoblastic leukemia cell line which harbors a heterozygous A687V EZH2 mutation was kindly provided by Dr. Ronald Levy (Stanford University) and was maintained in RPMI-1640 media (MediaTech) supplemented with 10% fetal bovine serum (FBS) (Sigma-Aldrich). All other cell lines were obtained from either ATCC or DSMZ and maintained in the recommended cell culture media.

### Example 5

### Transient Expression of WT and Mutant EZH2 Proteins in Cells

MCF-7 breast cancer cells (3×10⁵) were seeded into 6-well tissue culture plates in RPMI-1640 media supplemented with 10% FBS the day before transfection. Following the manufacturer's recommendations, 2 μg plasmid DNA and 6 μl Lipofectamine 2000 (Invitrogen) were combined in 500 μl Opti-MEM (Invitrogen) and incubated for 20 minutes at room temperature before being added to cells. Cells were then incubated for 72 hours at 37° C. with 5% CO₂ and harvested for protein lysates.

### Example 6

### Western Blot Analysis

Cell lysate preparation and western blotting were performed as previously described (McCabe et al., 2012a). Antibodies utilized included: EZH2 (BD Transduction Labs), histone H3 (Abcam), H3K27me1 (ActiveMotif), H3K27me2 (Cell Signaling Technology), H3K27me3 (Cell Signaling Technology), EED (Santa Cruz), SUZ12 (Cell Signaling Technology), or Actin (Sigma). Histone methylation antibody specificity was previously confirmed using full-length recombinant methylated histones harboring mono-, di-, or tri-methylation at H3K4, H3K9, H3K27, H3K36, and H3K79 (McCabe et al., 2012a).

### Example 7

### Sanger Sequencing of EZH2

Isolation of genomic DNA and Sanger sequencing of full-length EZH2, including exons 16 (Y641) and 18 (A677, A687), was performed as previously described (McCabe et al., 2012a).

### Example 8

### Cell Proliferation Assay

Cells were evaluated for sensitivity to GSK126 in a 6-day proliferation assay as described previously (McCabe et al., 2012b). Briefly, cells were seeded into 384 well plates at a density that permitted proliferation for 6 days. Cells were then treated in duplicate with a 20-point 2-fold dilution series of GSK126 or 0.147% DMSO. After incubation with compound for 6 days, cell proliferation was evaluated using CellTiter-Glo (Promega) according to the manufacturer's specifications. Data were fit with a 4-parameter equation to generate a concentration response curve and to determine the concentration of GSK126 required to inhibit 50% of growth (gIC₅₀).

### Example 9

### Caspase 3/7 Assay

For detection of caspase-3/7 activity, Caspase-Glo 3/7 (Promega) was utilized according to the manufacturer's directions. Values were normalized to CellTiter Glo (Promega) levels at each time point and expressed as a percentage of vehicle treated control.

### Example 10

### Gene Expression Profiling

SUP-B8 and NALM-6 cells (2×10⁵/well) were seeded into six-well tissue culture plates 24 hours before treatment with 0.1% DMSO or 500 nM GSK126 for 72 hours. Cells were collected into Trizol reagent (Invitrogen) and total RNA was isolated via phenol:chloroform extraction and the RNeasy kit (Qiagen) according to the manufacturer's instructions. Total RNA was labeled and hybridized to Affymetrix Human Genome U133 Plus 2.0 oligonucleotide microarrays arrays according to the manufacturer's instructions (Affymetrix, Santa Clara, Calif., USA). CEL files were processed and differentially expressed probe sets were determined as described previously (McCabe et al., 2012b). Briefly, significant probe sets were identified after filtering for detection, an average fold-change >2 or <-2, and p-values adjusted for multiple testing correction by FDR (Benjamini Hochberg)<0.1.

### Example 11

### Gene Ontology and Gene Set Enrichment Analysis (GSEA)

Functional analyses of differentially expressed probe sets were performed using DAVID (Huang et al., 2009). Significantly over-represented GO Biological Process and Molecular Function terms (levels 3-5) were filtered for terms with at least 5 significantly altered probes and EASE p-value <0.01. For GSEA (Subramanian et al., 2005), log₂ transformed microarray data were filtered (100% present calls and signal >6 in at least one cohort). In total, 25,059 probesets were included. Gene set permutations were used to identify significantly enriched gene sets from the c2 (curated gene sets) and c3 (motif gene sets) MSigDB V3.0 databases (Liberzon et al., 2011).

### Example 12

### EZH2 A687 is Mutated in Human Cancers and Affects Substrate Specificity

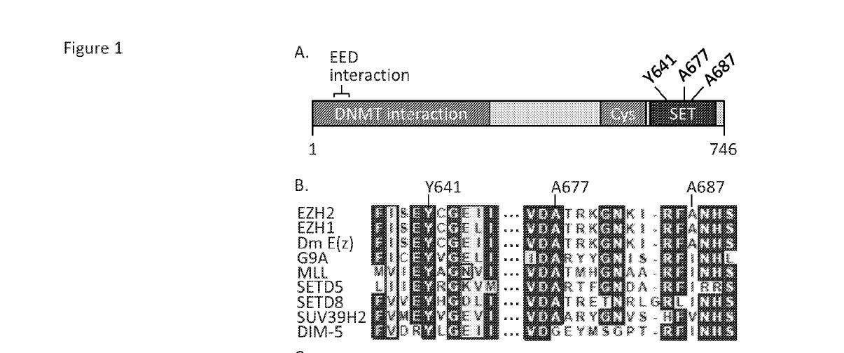

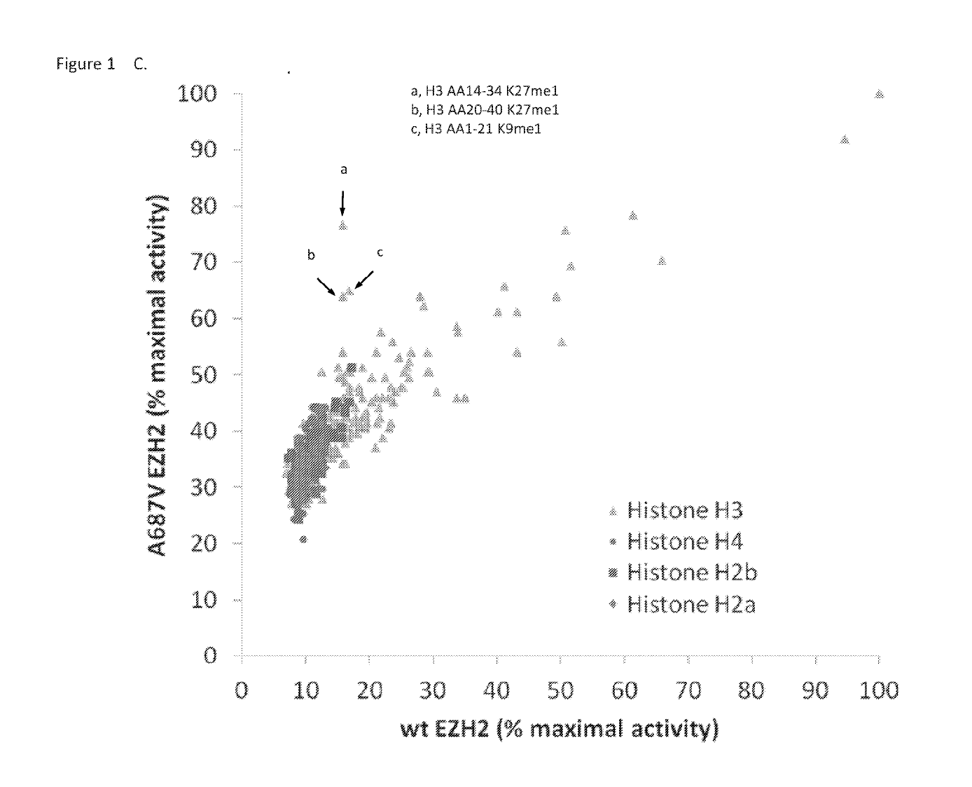

The A687V EZH2 mutation has been observed in two independent studies of DLBCLs with incidences of 1 of 49 and 1 of 127 (Lohr et al., 2012; Morin et al., 2011). While the incidence of this mutation appears to be quite low (˜1-2%), it is similar to what has been described for the A677G EZH2 mutation (McCabe et al., 2012a; Morin et al., 2011). A687 is located within the catalytic SET domain of EZH2 (FIG. 1A) and lies adjacent to the highly conserved NHS motif found in most SET domain methyltransferases (FIG. 1B). Interestingly, while the residues equivalent to EZH2 A687 are both alanine in human EZH1 and Drosophila E(z), this residue is most frequently either valine (31%) or isoleucine (35%) in other SET domain methyltransferases.

Recent biochemical studies (Majer et al., 2012) demonstrated that the A687V EZH2 exhibited 4-fold increased catalytic efficiency with a mono-methylated K27 substrate. To further evaluate whether this was the only change in substrate specificity, we assessed PRC2 complexes containing WT or A687V mutant EZH2 with a library of 602 peptides representing sequences within histones H2A, H2B, H3, or H4 and possessing up to five post-translational modifications, such as lysine and/or arginine methylation; lysine acetylation; or phosphorylation of serine, tyrosine, and/or threonine. Supporting the previous report (Majer et al., 2012), this global analysis revealed that A687V EZH2 exhibited enhanced activity for three peptides that showed little activity with WT EZH2 (FIG. 1C). These three peptides derived from histone H3 all harbored mono-methylation at either K27 (n=2) or K9 (n=1). Methylation of H3K9 containing peptides is a known biochemical artifact for the PRC2 complex that only occurs in vitro (Kuzmichev et al., 2002; Muller et al., 2002). Thus, the A687V EZH2 complex is unique from both WT and previously reported Y641 and A677 mutants in that it gains activity with a mono-methylated, and not di-methylated, substrate.

### Example 13

### Transient Expression of A687V EZH2 Increases Global H3K27me3 while Maintaining H3K27me2 Levels

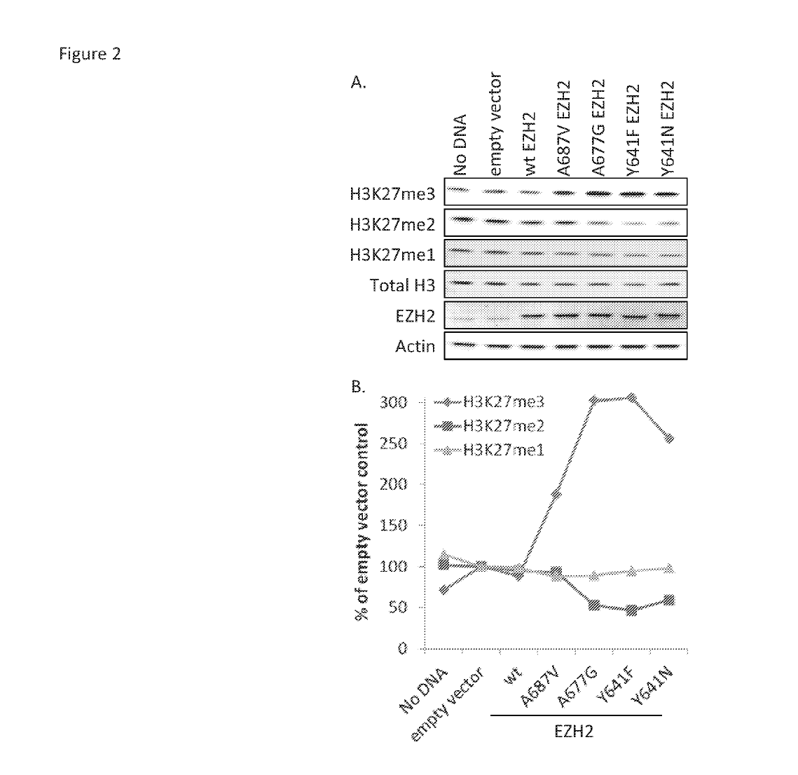

To explore the effect of the A687V EZH2 mutant on H3K27 methylation levels, WT and mutant versions of EZH2 were transiently expressed in MCF-7 cells. MCF-7 cells possess a WT EZH2 and have previously been shown to exhibit increased H3K27me3 and decreased H3K27me2 in response to transient expression of the Y641 and A677 EZH2 mutants (McCabe et al., 2012a). Interestingly, although A687V EZH2 exhibits increased efficiency in vitro for the di-methylation reaction, but not the tri-methylation reaction, exogenous expression of A687V EZH2 reproducibly increased H3K27me3 levels relative to the control (average 1.9-fold increase, n=7) (FIG. 2A/B). This represents approximately half the effect observed with the A677G, Y641F, or Y641N EZH2 mutants (average 3.0, 3.1, and 2.6-fold increases, n=10, 8, 8, respectively). However, in contrast to these mutants that induced 1.7-2.1 fold depletion of global H3K27me2 in the process of generating H3K27me3, A687V EZH2 maintained H3K27me2 at levels equivalent to that of WT EZH2 (average 1.1 fold decrease, n=5). H3K27me1 levels were not significantly affected by either WT or mutant EZH2. These observations suggest that the increased di-methylation activity of A687V EZH2 may increase H3K27me3 through the law of mass action and that over time equilibrium is achieved with increased H3K27me3 and near WT levels of H3K27me2.

### Example 14

### Identification of a B Cell Acute Lymphoblastic Leukemia Cell Line Harboring Heterozygous A687V EZH2

To identify a cell line possessing the A687V EZH2 mutation, a panel of cancer cell lines of B cell origin was sequenced for the A687 codon located in exon 18 of EZH2. A single cell line, SUP-B8, was identified to harbor a heterozygous A687V EZH2 mutation. The SUP-B8 cell line was established in 1988 from a 13 year old female with B cell acute lymphoblastic leukemia (Carroll et al., 1988). As this was to the best of our knowledge the first observation of a potentially activating EZH2 mutation in B cell ALL, we performed full-length sequencing of EZH2 in a panel of 11 B-cell ALL cell lines. Although no other A687V EZH2 mutations were identified, a heterozygous Y641N EZH2 mutation was identified in VAL, a cell line derived from the bone marrow of a 50 year old female diagnosed with B cell ALL (Dyer et al., 1996).

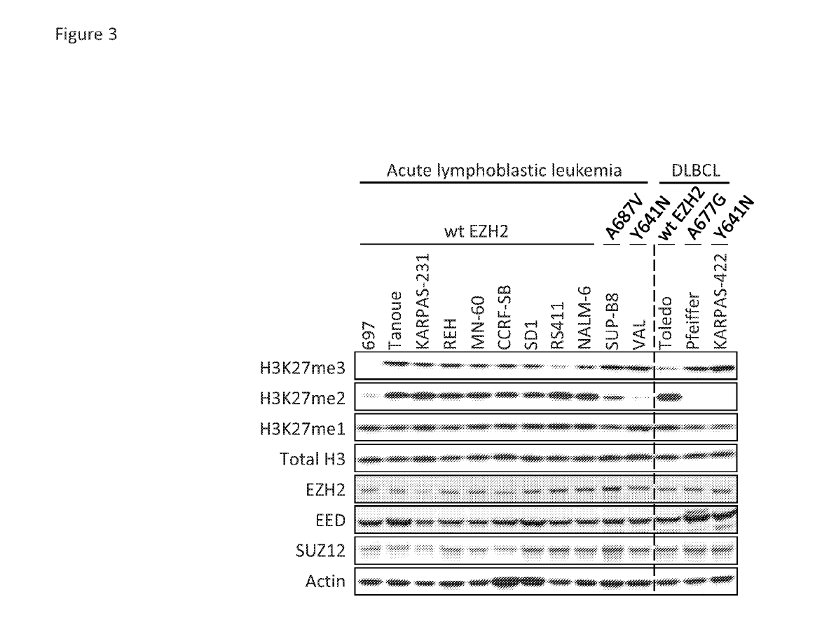

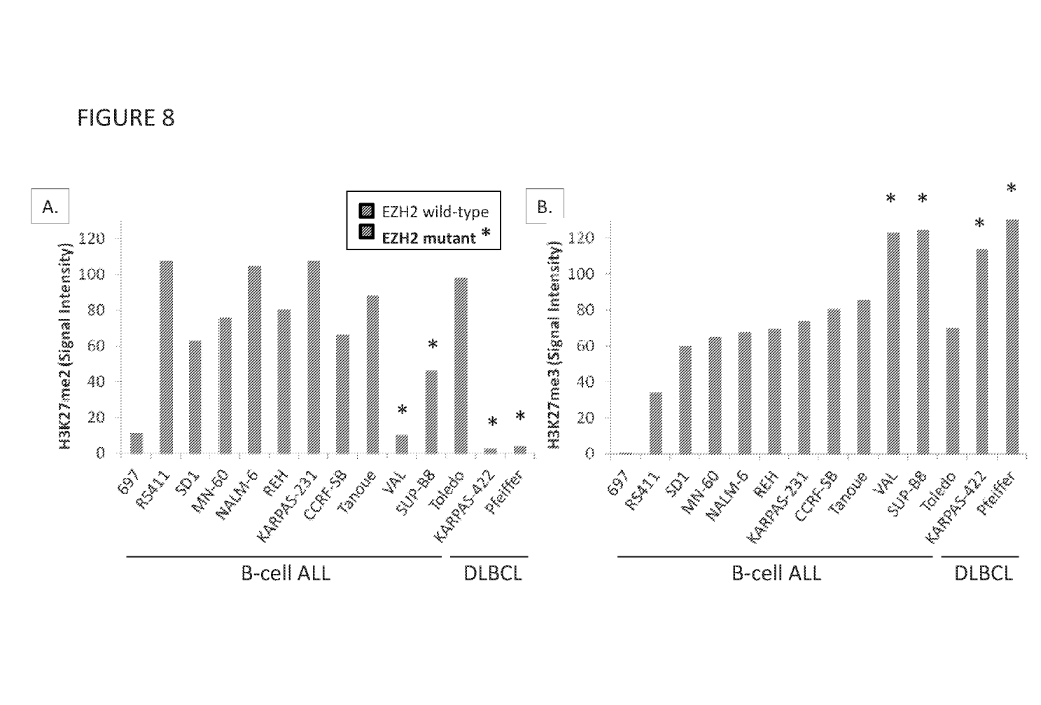

Since activating mutations at EZH2 Y641 and A677 in DLBCL cell lines lead to an imbalance of H3K27 methylation states with increased H3K27me3 and dramatically reduced H3K27me2 (McCabe et al., 2012a; Sneeringer et al., 2010), we examined the impact of the A687V and Y641N EZH2 mutations in the context of B cell ALL cell lines. Consistent with Y641 EZH2 mutant DLBCL cell lines, the Y641N EZH2 mutant VAL cell line exhibited elevated H3K27me3 and nearly complete loss of H3K27me2 (FIG. 3; FIG. 8). Interestingly, while the A687V EZH2 mutant SUP-B8 cell line exhibited high H3K27me3, its H3K27me2 levels were intermediate between those of EZH2 WT and Y641/A677 mutant cell lines. Thus, these findings from a panel of B cell ALL cell lines confirm the transient over-expression studies and demonstrate that A687V EZH2 promotes an imbalance in H3K27 methylation states different than Y641 or A677 EZH2 mutants.

### Example 15

### Cells Harboring the A687V EZH2 Mutation are Dependent Upon EZH2 Activity for Growth and Survival

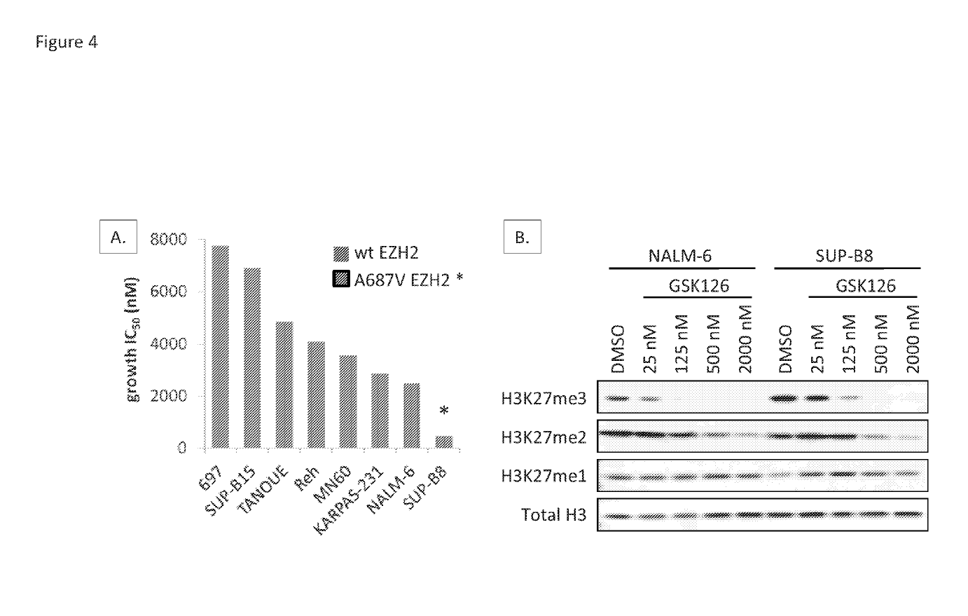

To examine the extent to which A687V EZH2 mutant cells are dependent upon EZH2 activity for their survival, we utilized a highly selective small molecule inhibitor of EZH2, GSK126. GSK126 has previously been shown to effectively inhibit the proliferation of many Y641 and A677 EZH2 mutant DLBCL cell lines (McCabe et al., 2012b). Among a panel of 8 B cell ALL cell lines, the A687V EZH2 mutant SUP-B8 cell line exhibited the greatest sensitivity to GSK126 with a dose of 441 nM being required to inhibit 50% of cell proliferation (growth IC₅₀) (FIG. 4A). EZH2 WT cell lines, on the other hand, exhibited a range of sensitivity to GSK126 with growth IC₅₀ values ranging from 2.5-7.8 This increased sensitivity observed for SUP-B8 in the proliferation assay likely reflects a greater dependence on EZH2 activity, and not a greater mechanistic potency of GSK126 in this cell line, as SUP-B8 exhibited comparable, or slightly reduced, demethylation of H3K27me3 when compared with the most sensitive EZH2 WT cell line, NALM-6 (FIG. 4B).

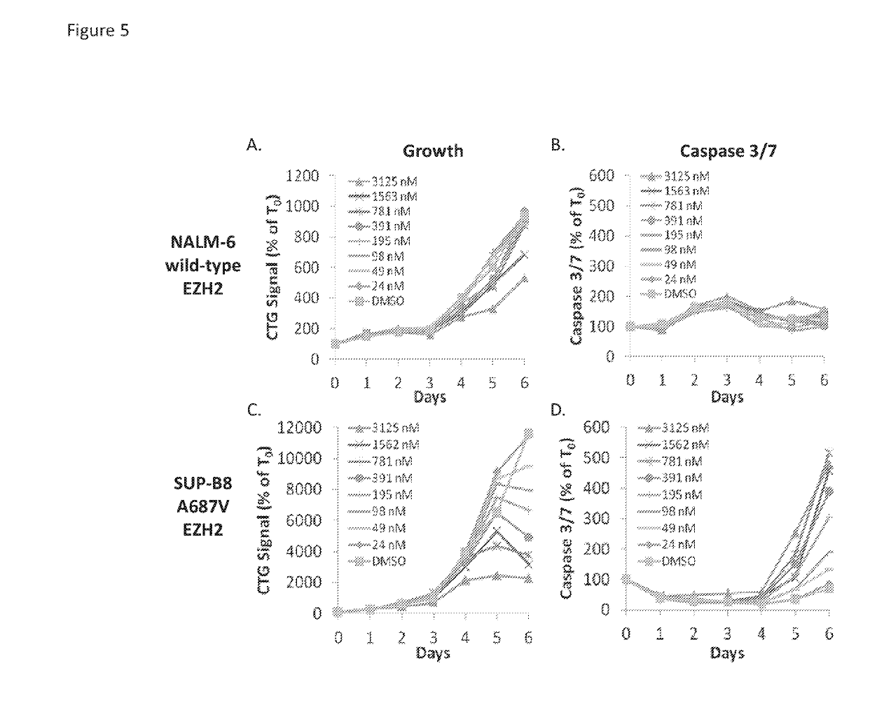

In order to evaluate the kinetics of growth inhibition and mechanism of cell death, SUP-B8 and NALM-6 cells were examined for cell proliferation and caspase 3/7 activity over a 6 day time course. While little growth inhibition and no activation of caspases 3 or 7 were observed out to 6 days in NALM-6 cells (FIG. 5A/B), a dose-dependent growth inhibition was observed in SUP-B8 cells after 4 days of treatment and was maximal on day 6 when cell number losses were evident for all doses above 100 nM GSK126 suggesting induction of cell death mechanisms (FIG. 5C). Consistent with these observations, caspase 3/7 activity was increased in a dose-dependent fashion beginning on day 5 (FIG. 5D) demonstrating that GSK126 potently induces apoptotic cell death in A687V EZH2 mutant cells.

### Example 16

### EZH2 Inhibition Elicits Transcriptional Activation of Silenced Genes in the A687V EZH2 Mutant Cell Line

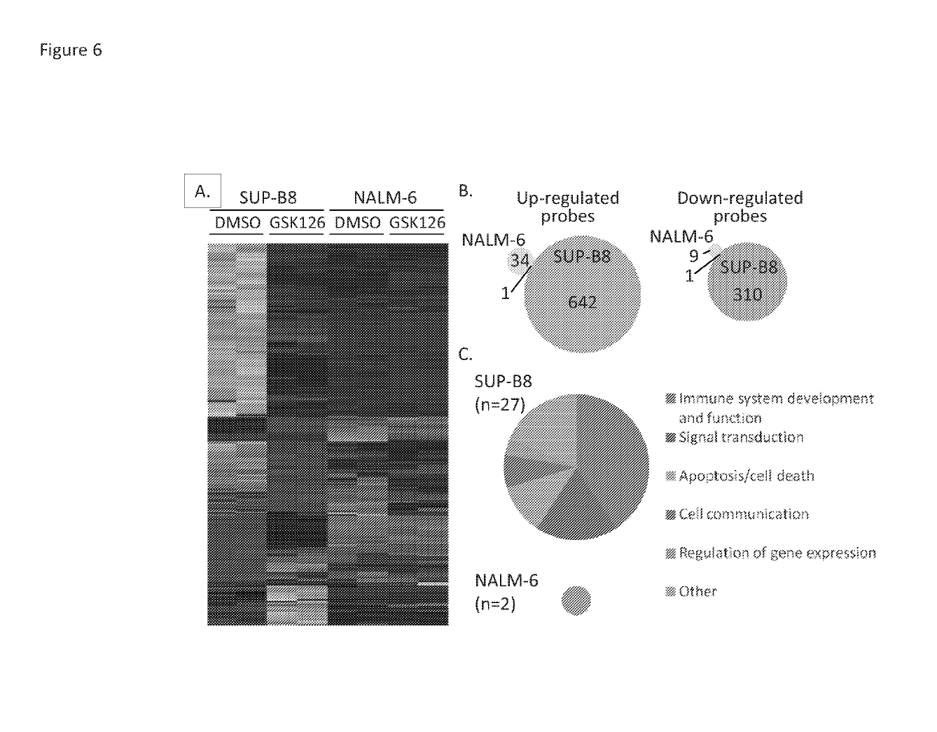

Previous studies have demonstrated that while both sensitive and resistant lymphoma cell lines lose H3K27me3 with nearly equal potency in response to treatment inhibition of EZH2, only sensitive cell lines exhibit a robust program of transcriptional activation (McCabe et al., 2012b). When NALM-6 cells were treated with 500 nM GSK126 for 72 hours, minimal transcriptional effects were observed with only 35 up-regulated probes and 10 down-regulated probes (FIG. 6A/B). SUP-B8, on the other hand, exhibited marked transcriptional activation with 643 up-regulated probes (FIG. 6A/B). Analysis of gene ontology terms enriched among the significantly changed probes from SUP-B8 cells suggests regulation of genes involved several pathways including immune system development and function (e.g. AICDA, CXCR5, IL7, IL15, LCK, LTA, LTB, STAT5A, TNF) and apoptosis (e.g. BCL2A1, BCL2L1, BCL2L11, CASP2, CFLAR, PDCD4, TNF, TNFRSF1A, TNFSF10, TNFRSF21, TRADD) whereas NALM-6 only exhibited enrichment of two apoptosis-related terms (FIG. 6C). Gene Set Enrichment Analysis (GSEA) additionally revealed an enrichment of genes involved in lymphocyte and plasma cell differentiation, interferon response, and apoptosis among the genes that were significantly up-regulated in the SUP-B8 cell line. Thus, inhibition of EZH2 catalytic activity and loss of H3K27me3 in A687V EZH2 cells induces a program of transcriptional activation for genes associated with lymphocyte activation and cell death suggesting a greater dependence on EZH2 in A687V mutant versus WT cells.

### Example 17

Examination of Y641 and A677 EZH2 mutants suggests that alterations to the lysine binding pocket architecture can affect substrate preferences leading to global hyper-trimethylation of H3K27 (McCabe et al., 2012a; Sneeringer et al., 2010; Yap et al., 2011). In addition to these two mutated residues, numerous mutations have now been reported throughout the entire EZH2 coding sequence. Most of these mutations observed in myeloid cancers, however, appear to inactivate enzymatic activity likely reflecting a tumor suppressive role for EZH2 and/or PRC2 in myeloid cells (Hock, 2012). It is necessary therefore to consider that EZH2 mutations may be activating or inactivating and that a thorough understanding of the biochemical and cellular consequences of each mutation may help clarify the oncogenic or tumor suppressive activities of EZH2 in different contexts. To this end, we have characterized the cellular effects of A687V EZH2 and demonstrate that this mutant increases global H3K27me3, but unlike Y641 and A687V EZH2 mutants, retains near normal levels of H3K27me2. Importantly, these cells appear to be highly dependent on EZH2 activity for their survival.

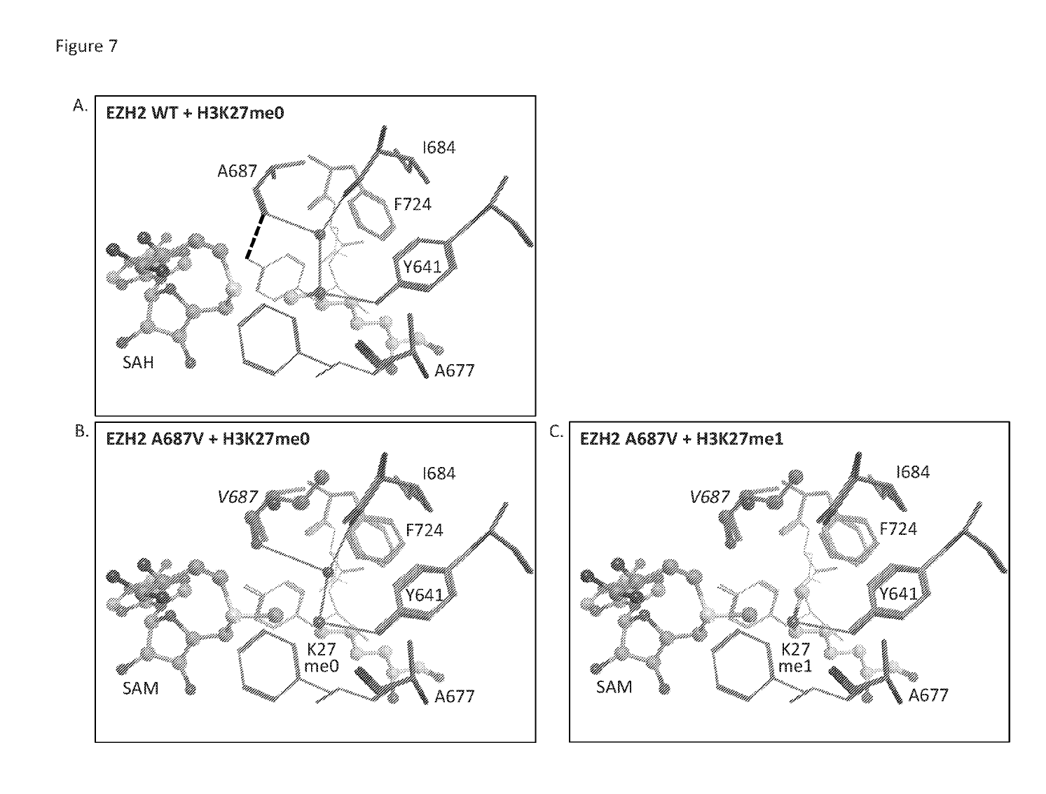

Since EZH2 and PRC2 crystal structures have remained elusive, a homology model of the EZH2 SET domain was constructed to understand the location of A687 within the catalytic domain. This previously described model is based on the protein sequence of EZH2 and a crystal structure of GLP/EHMT1 bound to a histone H3 peptide containing H3K9me2 (McCabe et al., 2012a). In this model, A687 resides at the interface of the SAM and peptide binding pockets (FIG. 7A). The A687 side chain is buried from solvent and points away from the SAM pocket toward F724, the phenylalanine-tyrosine switch residue of EZH2. The backbone carbonyl of A687 points into the active site and forms H-bonds with both Y726 and a conserved active site water molecule. While Y726 appears to contribute to efficient binding of SAM/SAH as well as proper folding of the SET domain, the active site water molecule is stabilized by the carbonyl groups of A687 and 1684 and likely helps coordinate the lysine substrate. Thus, A687 appears to occupy a highly conserved position within the SET domain and may function in multiple roles to regulate binding of both SAM and lysine substrates.

Based upon study of our EZH2 active site model, related SET domain containing methyltransferases, and various SET domain mutants, different enzyme:substrate interactions appear to be required for mono-, di-, and tri-methylation reactions. For the me0→me1 reaction, the key requirement appears to be proper positioning of the lysine substrate's ε-amine at the methyl transfer pore. This is accomplished in large part by the highly conserved tyrosine residue (Y641 in EZH2, Y245 in SET7/9;) and the active site water molecule as evidenced by the loss of activity with unmethylated lysine substrates in the EZH2 Y641N/F/S/H/C and SET7/9 Y245A mutants (Del Rizzo et al., 2010; McCabe et al., 2012a; Sneeringer et al., 2010; Yap et al., 2011). For the me1→me2 reaction, the lysine substrate must be oriented with the Kme1 methyl group rotated away from the methyl transfer pore so that a second methyl group may be transferred. This requires that the conserved active site water molecule be displaced from the lysine binding pocket or relocated within the enzyme to make room for the larger substrate. Finally, the me2→me3 reaction is accomplished when the Kme2 substrate is oriented with both methyl groups positioned away from the methyl transfer pore. This step is limited by steric hinderance created between the large Kme2 substrate and EZH2 Y641, SET7/9 Y245, or G9a Y1067. Mutation of this restrictive tyrosine to a smaller residue converts these enzymes into efficient tri-methyltransferases (Del Rizzo et al., 2010; McCabe et al., 2012a; Sneeringer et al., 2010; Wu et al., 2010; Yap et al., 2011).

The active site water molecule in EZH2 appears to be coordinated by A687, 1684, and the lysine substrate (FIG. 7A). In SET domain enzymes where the F/Y switch residue is a tyrosine, the hydroxyl group of the tyrosine creates a fourth stabilizing interaction for this water (see SET8 and SET7/9). Based on the role of the active site water molecule in regulating product specificity and the positioning of this water in part by EZH2 A687, we propose that mutation of A687 to the larger valine residue results in changes to the lysine substrate pocket such that binding of the water is destabilized relative to the WT enzyme. In this scenario, it would be easier for the methyl group of the H3K27me1 substrate to be accommodated in the active site of A687V EZH2 through displacement of the less tightly bound water thereby enhancing turnover with an H3K27me1 substrate.

Although there has not been much work focused on EZH2 A687, or the equivalent residue in other enzymes, this proposal is supported by extensive work with SET domain F/Y switch residues that also coordinate the active site water molecule. In SET7/9, this residue is a tyrosine (Y305) and mutation to a phenylalanine increases the amount of Kme2 product (Del Rizzo et al., 2010). Conversely, in G9A this residue is a phenylalanine (F1152) and mutation to a tyrosine reduces the amount of Kme2 product. The mechanism by which the F/Y switch residue affects product specificity is through the strength of hydrogen bonding to the active site water molecule. When a tyrosine is in the F/Y switch position, the phenolic OH group forms an H-bond to the water, resulting in the inability to accommodate the methyl group of a monomethyl lysine substrate. When a phenylalanine occupies this position, the water molecule is bound less tightly and can be displaced resulting in the increased ability to form Kme2 product. Thus, even though EZH2 contains a phenylalanine in the switch position and can accomplish di-methylation, the A687V mutation appears to further weaken the binding of the active site water and facilitates a further increase in the rate of di-methylation.

Despite the fact that the predominant biochemical alteration is a gain in activity with an H3K27me1 substrate (Majer et al., 2012), when A687V EZH2 is transiently expressed in cells or found to occur naturally in an ALL cell line, the observed phenotype is increased tri-methylation of H3K27, and not increased di-methylation (FIGS. 2/3). The data presented herein and in the literature suggest that the Y641, A677, and A687 EZH2 mutants all stimulate hyper-trimethylation of H3K27, but vary in the extent to which they induce hypo-dimethylation of H3K27. Unfortunately, the relevance of this difference is unknown since little is understood about the distinct roles played by H3K27me2 and H3K27me3. Global studies of histone marks from CD4+ T cells suggests that while H3K27me1 is enriched at actively transcribed genes, both H3K27me2 and H3K27me3 are present at genes that are silenced or lowly expressed (Barski et al., 2007). Work from Reinberg and colleagues suggests that H3K27me2 is associated with lowly expressed or poised genes, while the conversion to H3K27me3 may more completely silence gene expression (Sarma et al., 2008). Interestingly, the transition from di- to tri-methylation appears to be stimulated by the presence of PHF1, a Polycomblike (Pcl) family member, known to co-immunoprecipitate with a fraction of cellular PRC2 (Cao et al., 2008; Nekrasov et al., 2007; Sarma et al., 2008). The Drosophila Pcl protein has three human homologs including PHF1, MTF2, and PHF19 and all include at least one PHD finger domain and a TUDOR domain. The existence of specific biological mechanisms for the regulation of H3K27me2 and H3K27me3 suggests that there may be distinct biological requirements for these two marks and perhaps study of these EZH2 mutant cell lines can provide some insight to their functions.

Considering that mutations of EZH2 Y641 are found in up to 22% GCB DLBCL and FL, it is somewhat surprising that mutations of A677 and A687 appear to be so rare (˜1-2%) since they too increase H3K27me3. We have previously proposed for the A677G EZH2 mutation that this is due in part to the fact that glycine appears to be the only amino acid that when substituted for A677 will promote hyper-trimethylation and that when considering single nucleotide mutations of the A677 codon only a single mutation is capable of generating a glycine (McCabe et al., 2012a). The low incidence of the A687V EZH2 mutation may be similarly explained. At the nucleotide level, only one of nine single-nucleotide mutations within the A687 codon will result in a valine residue. Other amino acids that can be generated by single nucleotide mutations include glutamic acid, proline, threonine, serine, and glycine. We hypothesize that glutamic acid and proline mutations may disrupt proper enzyme folding due to their dramatically different properties when compared to the WT alanine. Serine and threonine may be tolerated based on their size alone; however, the polar side chains of these residues might also alter enzyme folding. Lastly, glycine, a slightly smaller residue, may be tolerated, but may not change the wild-type product specificity as the active site water molecule would likely still be sufficiently coordinated with this substitution. Therefore, it appears that the frequency of these mutations may relate to multiple independent factors that can be rationalized when placed within the context of the enzyme's active site and the various interactions required for proper functioning.

While activating mutations in EZH2 (Y641F/N/S/H/C, A677G, A687V) have thus far been found primarily in GCB DLBCL and FL (Bodor et al., 2011; Lohr et al., 2012; McCabe et al., 2012a; Morin et al., 2010; Morin et al., 2011; Ryan et al., 2011), this study identified cases of Y641N and A687V EZH2 mutations in B cell ALL cell lines. While much additional screening is required to determine the incidence of these mutations in B-cell ALL, this observation raises the question whether EZH2 may be uniquely required for homeostasis in the cell types from which these tumors arise. GCB DLBCL and FL arise from centroblasts or centrocytes, respectively (Nogai et al., 2011), and common B-cell ALL arises from pre-pro-B cells or pro-B cells (Cobaleda and Sanchez-Garcia, 2009). EZH2 expression appears to be tightly regulated throughout B-cell differentiation. EZH2 levels are high in pro-B cells and then decrease in pre-B cells and are nearly undetectable in immature naïve B cells (Su et al., 2003; Velichutina et al., 2010). EZH2 is then up-regulated during the germinal center reaction in centrocytes and centroblasts before decreasing again in mature re-circulating B-cells (Su et al., 2003; Velichutina et al., 2010). Thus, it appears that activating mutations of EZH2 are found in cancer cells arising from cell types that highly express EZH2. This may suggest that these tumors select for cells harboring pre-existing activating mutations in EZH2 since they provide an opportunity for the mutant EZH2 to be expressed. Alternatively, it may be that silencing of EZH2 is critical for progression to the next stage of differentiation and that activated EZH2 establishes an altered epigenetic state that impedes further differentiation and promotes transformation in the presence of additional oncogenic mutations. Further work with mouse models engineered to over-express mutant EZH2 at various stages of B-cell differentiation may provide insight to these critical questions.

This study has demonstrated that the A687V EZH2 mutation is capable of increasing global H3K27me3 in cells and that a cancer cell line harboring this mutation is dependent on EZH2 activity for its survival. Thus, there are now three EZH2 residues, that when mutated in human cancers, alter the default substrate preferences leading to aberrant post-translational modification of histone tails. Our studies with a specific EZH2 inhibitor demonstrate that targeting of EZH2 in these mutant cell lines is an efficient strategy for inducing growth inhibition and cell killing. These studies and others (Knutson et al., 2012; McCabe et al., 2012b; Qi et al., 2012) provide encouraging data supporting the use of EZH2 inhibitors in patients harboring activating mutations of EZH2.

### REFERENCES

Barski A, Cuddapah S, Cui K, Roh T Y, Schones D E, Wang Z et al (2007). High-resolution profiling of histone methylations in the human genome. Cell 129: 823-37.
Bodor C, O'Riain C, Wrench D, Matthews J, lyengar S, Tayyib H et al (2011). EZH2 Y641 mutations in follicular lymphoma. Leukemia 25: 726-9.
Bracken A P, Dietrich N, Pasini D, Hansen K H, Helin K (2006). Genome-wide mapping of Polycomb target genes unravels their roles in cell fate transitions. Genes Dev 20: 1123-36.
Bracken A P, Pasini D, Capra M, Prosperini E, Colli E, Helin K (2003). EZH2 is downstream of the pRB-E2F pathway, essential for proliferation and amplified in cancer. EMBO J 22: 5323-35.
Cao R, Wang H, He J, Erdjument-Bromage H, Tempst P, Zhang Y (2008). Role of hPHF1 in H3K27 methylation and Hox gene silencing. Mol Cell Biol 28: 1862-72.
Cao R, Wang L, Wang H, Xia L, Erdjument-Bromage H, Tempst P et al (2002). Role of histone H3 lysine 27 methylation in Polycomb-group silencing. Science 298: 1039-43.
Carroll W L, Link M P, Cleary M L, Bologna S, Carswell C, Amylon M D et al (1988). Idiotype as a tumor-specific marker in childhood B cell acute lymphoblastic leukemia. Blood 71: 1068-73.
Cobaleda C, Sanchez-Garcia I (2009). B-cell acute lymphoblastic leukaemia: towards understanding its cellular origin. Bioessays 31: 600-9.
Del Rizzo P A, Couture J F, Dirk L M, Strunk B S, Roiko M S, Brunzelle J S et al (2010). SET7/9 catalytic mutants reveal the role of active site water molecules in lysine multiple methylation. J Biol Chem 285: 31849-58.
Dyer M J, Lillington D M, Bastard C, Tilly H, Lens D, Heward J M et al (1996). Concurrent activation of MYC and BCL2 in B cell non-Hodgkin lymphoma cell lines by translocation of both oncogenes to the same immunoglobulin heavy chain locus. Leukemia 10: 1198-208.
Findeis-Holey J J, Huang J, Li F, Yang Q McMahon L A, Xu H (2011). High-grade neuroendocrine carcinomas of the lung highly express enhancer of zeste homolog 2, but carcinoids do not. Hum Pathol 42: 867-72.
Hock H (2012). A complex Polycomb issue: the two faces of EZH2 in cancer. Genes Dev 26: 751-5.
Huang D W, Sherman B T, Lempicki R A (2009). Systematic and integrative analysis of large gene lists using DAVID bioinformatics resources. Nat Protoc 4: 44-57.
Kirmizis A, Bartley S M, Kuzmichev A, Margueron R, Reinberg D, Green R et al (2004). Silencing of human polycomb target genes is associated with methylation of histone H3 Lys 27. Genes Dev 18: 1592-605.
Kleer C G, Cao Q, Varambally S, Shen R, Ota I, Tomlins S A et al (2003). EZH2 is a marker of aggressive breast cancer and promotes neoplastic transformation of breast epithelial cells. Proc Natl Acad Sci USA 100: 11606-11.
Knutson S K, Wigle T J, Warholic N M, Sneeringer C J, Allain C J, Klaus C R et al (2012). A selective inhibitor of EZH2 blocks H3K27 methylation and kills mutant lymphoma cells. Nat Chem Biol 8: 890-6.
Kuzmichev A, Nishioka K, Erdjument-Bromage H, Tempst P, Reinberg D (2002). Histone methyltransferase activity associated with a human multiprotein complex containing the Enhancer of Zeste protein. Genes Dev 16: 2893-905.
Liberzon A, Subramanian A, Pinchback R, Thorvaldsdottir H, Tamayo P, Mesirov J P (2011). Molecular signatures database (MSigDB) 3.0. Bioinformatics 27: 1739-40.
Lohr J G, Stojanov P, Lawrence M S, Auclair D, Chapuy B, Sougnez C et al (2012). Discovery and prioritization of somatic mutations in diffuse large B-cell lymphoma (DLBCL) by whole-exome sequencing. Proc Natl Acad Sci USA 109: 3879-84.
Majer C R, Jin L, Scott M P, Knutson S K, Kuntz K W, Keilhack H et al (2012). A687V EZH2 is a gain-of-function mutation found in lymphoma patients. FEBS Lett 586: 3448-51.
McCabe M T, Graves A P, Ganji G, Diaz E, Halsey W S, Jiang Y et al (2012a). Mutation of A677 in histone methyltransferase EZH2 in human B-cell lymphoma promotes hypertrimethylation of histone H3 on lysine 27 (H3K27). Proc Natl Acad Sci USA 109: 2989-94.
McCabe M T, Ott H M, Ganji G, Korenchuk S, Thompson C, Van Aller G S et al (2012b). EZH2 inhibition as a therapeutic strategy for lymphoma with EZH2-activating mutations. Nature.
Morin R D, Johnson N A, Severson T M, Mungall A J, An J, Goya R et al (2010). Somatic mutations altering EZH2 (Tyr641) in follicular and diffuse large B-cell lymphomas of germinal-center origin. Nat Genet 42: 181-5.
Morin R D, Mendez-Lago M, Mungall A J, Goya R, Mungall K L, Corbett R D et al (2011). Frequent mutation of histone-modifying genes in non-Hodgkin lymphoma. Nature 476: 298-303.
Muller J, Hart C M, Francis N J, Vargas M L, Sengupta A, Wild B et al (2002). Histone methyltransferase activity of a Drosophila Polycomb group repressor complex. Cell 111: 197-208.
Nekrasov M, Klymenko T, Fraterman S, Papp B, Oktaba K, Kocher T et al (2007). Pcl-PRC2 is needed to generate high levels of H3-K27 trimethylation at Polycomb target genes. EMBO J 26: 4078-88.
Nogai H, Dorken B, Lenz G (2011). Pathogenesis of non-Hodgkin's lymphoma. J Clin Oncol 29: 1803-11.
Qi W, Chan H, Teng L, Li L, Chuai S, Zhang R et al (2012). Selective inhibition of Ezh2 by a small molecule inhibitor blocks tumor cells proliferation. Proc Natl Acad Sci USA 109: 21360-5.
Ryan R J, Nitta M, Borger D, Zukerberg L R, Ferry J A, Harris N L et al (2011). EZH2 codon 641 mutations are common in BCL2-rearranged germinal center B cell lymphomas. PLoS One 6: 1-7.
Sander S, Bullinger L, Klapproth K, Fiedler K, Kestler H A, Barth T F et al (2008). MYC stimulates EZH2 expression by repression of its negative regulator miR-26a. Blood 112: 4202-12.
Saramaki O R, Tammela T L, Martikainen P M, Vessella R L, Visakorpi T (2006). The gene for polycomb group protein enhancer of zeste homolog 2 (EZH2) is amplified in late-stage prostate cancer. Genes Chromosomes Cancer 45: 639-45.
Sarma K, Margueron R, Ivanov A, Pirrotta V, Reinberg D (2008). Ezh2 requires PHF1 to efficiently catalyze H3 lysine 27 trimethylation in vivo. Mol Cell Biol 28: 2718-31.
Simon J A, Lange C A (2008). Roles of the EZH2 histone methyltransferase in cancer epigenetics. Mutat Res 647: 21-9.
Sneeringer C J, Scott M P, Kuntz K W, Knutson S K, Pollock R M, Richon V M et al (2010). Coordinated activities of wild-type plus mutant EZH2 drive tumor-associated hypertrimethylation of lysine 27 on histone H3 (H3K27) in human B-cell lymphomas. Proc Natl Acad Sci USA 107: 20980-5.
Su I H, Basavaraj A, Krutchinsky A N, Hobert O, Ullrich A, Chait B T et al (2003). Ezh2 controls B cell development through histone H3 methylation and lgh rearrangement. Nat Immunol 4: 124-31.
Subramanian A, Tamayo P, Mootha V K, Mukherjee S, Ebert B L, Gillette M A et al (2005). Gene set enrichment analysis: a knowledge-based approach for interpreting genome-wide expression profiles. Proc Natl Acad Sci USA 102: 15545-50.
Takawa M, Masuda K, Kunizaki M, Daigo Y, Takagi K, Iwai Y et al (2011). Validation of the histone methyltransferase EZH2 as a therapeutic target for various types of human cancer and as a prognostic marker. Cancer Sci 102: 1298-305.
Varambally S, Cao Q, Mani R S, Shankar S, Wang X, Ateeq B et al (2008). Genomic loss of microRNA-101 leads to overexpression of histone methyltransferase EZH2 in cancer. Science 322: 1695-9.
Varambally S, Dhanasekaran S M, Zhou M, Barrette T R, Kumar-Sinha C, Sanda M G et al (2002). The polycomb group protein EZH2 is involved in progression of prostate cancer. Nature 419: 624-9.
Velichutina I, Shaknovich R, Geng H, Johnson N A, Gascoyne R D, Melnick A M et al (2010). EZH2-mediated epigenetic silencing in germinal center B cells contributes to proliferation and lymphomagenesis. Blood 116: 5247-55.
Wagener N, Macher-Goeppinger S, Pritsch M, Husing J, Hoppe-Seyler K, Schirmacher P et al (2010). Enhancer of zeste homolog 2 (EZH2) expression is an independent prognostic factor in renal cell carcinoma. BMC Cancer 10: 1-10.
Wu H, Min J, Lunin V V, Antoshenko T, Dombrovski L, Zeng H et al (2010). Structural biology of human H3K9 methyltransferases. PLoS One 5: e8570.
Yap D B, Chu J, Berg T, Schapira M, Cheng S W, Moradian A et al (2011). Somatic mutations at EZH2 Y641 act dominantly through a mechanism of selectively altered PRC2 catalytic activity, to increase H3K27 trimethylation. Blood 117: 2451-9.

1. A method of treating cancer in a human in need thereof, comprising determining at least one of the following in one or more samples from said human:
a. the presence or absence of an alanine to valine mutation at residue 687 (A687V) in EZH2 in a sample from said human; or
b. the presence or absence of an increased level of H3K27me2 in a sample from said human as compared to a control;
and administering to said human an effective amount of an EZH2 inhibitor or a pharmaceutically acceptable salt thereof if the A687V mutation is present, or an increased level of H3K27me2 is not present, or both, in the one or more samples, wherein the EZH2 inhibitor is a compound of Formula (I): 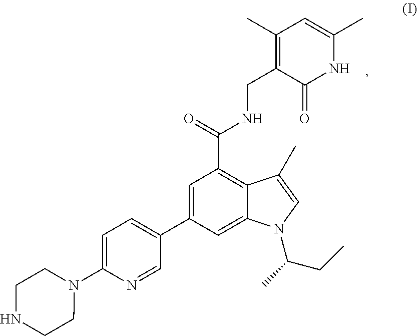 [SMILES: CC[C@H](C)n1cc(C)c2c(C(=O)NCc3c(C)cc(C)[nH]c3=O)cc(-c3ccc(N4CCNCC4)nc3)cc21]
or a pharmaceutically acceptable salt thereof.

2. The method of claim 1, wherein both a and b are determined.

3. The method of claim 1, further comprising determining the level of global H3K27me3 in a sample from the human as compared to a control and only administering the EZH2 inhibitor of Formula I or pharmaceutically acceptable salt thereof if the level of global H3K27me3 in the sample is elevated relative to a control, and if the A687V mutation is present, or an increased level of H3K27me2 is not present, or both, in said sample.

4. The method of claim 1, wherein
said human having one or more samples determined to have a presence of an A687V mutation in EZH2, or an absence of an increased level of H3K27me2 as compared to a control, or both, has an increased response rate and/or an improved progression free survival when treated with the EZH2 inhibitor of Formula I or a pharmaceutically acceptable salt thereof as compared to a human without a mutation in EZH2.

5. The method of claim 1, wherein said human having one or more samples determined to have a presence of an A687V mutation in EZH2, or an absence of an increased level of H3K27me2 as compared to a control, or both, additionally has a sample determined to have an increased overall level of H3K27me3 as compared to a control, and said human has an increased response rate, an improved progression free survival, or both, when treated with the EZH2 inhibitor of Formula I or a pharmaceutically acceptable salt thereof as compared to a human without a mutation in EZH2.

6. The method of claim 1, further comprising administering one or more additional anti-neoplastic agents.

7. The method of claim 1, wherein each of the one or more samples comprises at least one cancer cell.

8. The method of claim 1, wherein the A687V mutation in EZH2 is a somatic mutation.

9. The method of claim 1, wherein the cancer is lymphoma.

10. The method of claim 9, wherein the lymphoma is selected from the group consisting of: B-cell acute lymphoblastic leukemia (ALL), germinal center B-cell (GCB), Diffuse Large B-cell Lymphoma (DLBCL), Splenic marginal zone lymphoma (SMZL), Waldenström's macroglobulinemia lymphoplasmacytic lymphoma (WM), Follicular lymphoma (FL), Mantle Cell Lymphoma (MCL), and Extra nodal marginal zone B-cell lymphoma of mucosa associated lymphoid tissue (MALT).

11. The method of claim 9, wherein the lymphoma is B-cell acute lymphoblastic leukemia (ALL).

12. A kit for the treatment of cancer comprising a kit for determining one, two or three of a, b, and c:
a. the presence or absence of an alanine to valine mutation at residue 687(A687V) in EZH2 in a sample from said human;
b. the presence or absence of an increased level of H3K27me2 as compared to a control;
c. the level of global H3K27me3 in a sample from the human as compared to a control,
a means for determining one, two, or three of a, b, and c, and instructions for administering an EZH2 inhibitor, wherein said EZH2 inhibitor is an inhibitor of Formula I: 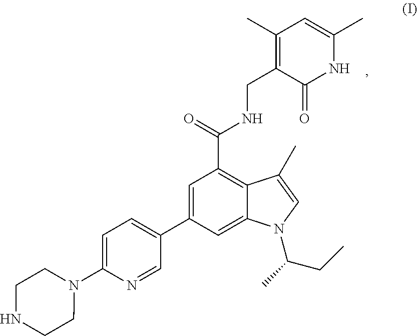 [SMILES: CC[C@H](C)n1cc(C)c2c(C(=O)NCc3c(C)cc(C)[nH]c3=O)cc(-c3ccc(N4CCNCC4)nc3)cc21]
or a pharmaceutically acceptable salt thereof, and wherein said instructions are in accordance with the method of claim 1.

13. The kit of claim 12, wherein said means is selected from the group consisting of primers, probes, and antibodies.

14-17. (canceled)
